(19) United States
(12) Patent Application Publication
(10) Pub. No.: US 2024/0067990 A1
(43) Pub. Date: Feb. 29, 2024

(54) POLYMERIC MICELLE COMPOSITIONS

(71) Applicant: UVic Industry Partnerships Inc.- Univ. of Victoria, Victoria (CA)

(72) Inventors: MATTHEW MOFFIT, VICTORIA (CA); SUNDIATA KLY, VICTORIA (CA)

(21) Appl. No.: 18/238,074

(22) Filed: Aug. 25, 2023

Related U.S. Application Data
(60) Provisional application No. 63/401,524, filed on Aug. 26, 2022.

(51) Int. Cl. C12N 15/88

(52) U.S. Cl. CPC C12N 15/88

(57) ABSTRACT

The present invention relates to polymeric micelle compositions. More specifically, the present invention relates to polymeric micelle compositions for gene delivery.

### FIELD

The present invention relates to polymeric micelle compositions. More specifically, the present invention relates to polymeric micelle compositions for gene delivery.

### BACKGROUND

Gene therapy opens up new routes for the treatment of chronic disease by modulating gene expression in targeted cells through the delivery of nucleic acids.¹ Although viral delivery vectors can lead to high transfection efficiencies in animal models, their clinical applications may be limited by potential immunogenicity and mutagenesis.^{2,3} In addition, the limited size capacity of viral particles may preclude the delivery of larger nucleic acids useful for some gene therapies⁴. Therefore, nonviral vectors have been investigated for gene delivery.^{2,3,5-11}

Polymer nanoparticles (or PNPs) are a type of nonviral gene delivery vectors and a wide range of such polymeric systems have been developed.¹²⁻¹⁶ Simple polymeric systems for gene delivery are polycations capable of electrostatic binding with negatively charged nucleic acids, forming polymer-nucleic acid complexes (polyplexes).¹³ A large number of polymers with linear, branched, and dendritic architectures have been applied in this manner, including polyethylenimine (PEI), poly(L-lysine) (PLL), and poly-(amido-amine) (PAMAM).^{13,15} Chemical modification of the simple polycation motif may improve gene delivery properties. For example, attaching poly-(ethylene glycol) (PEG) chains to polyplexes (PEGylation) can form a hydrophilic shell layer surrounding the polyplex, which has been implicated in increased blood dispersibility and lowered cytotoxicity.¹³ Moreover, PEGylation may increase nucleic acid resistance to nuclease degradation, by sterically inhibiting penetration of nucleases to the polyplex core.¹⁷

The installation of hydrophobic groups onto a polycation framework has been used to improve the properties of gene delivery PNPs.^{8, 17, 19, 20} When these hydrophobic moieties are relatively small (i.e., nonpolymeric), such as alkyl chains or cholesterol groups, the resulting hydrophobic interactions in water give rise to increased polyplex stability and nuclease resistance.^{17,19} Enhanced gene delivery capabilities can be realized when the hydrophobic groups are polymeric chains covalently attached to polyplex-forming polycations within a block copolymer architecture.^{4,14,17-19,21-29} Self-assembly of these block copolymers in aqueous environments gives rise to PNPs (termed “polyplex micelles”) in which hydrophobic chains compartmentalize into discrete hydrophobic domains segregated from the polycation-nucleic acid polyplex. In the vast majority of block copolymer-based gene delivery vehicles, hydrophobic blocks (usually biodegradable polyesters, including poly(lactic acid) (PLA), poly(lactic-co-glycolic acid) (PLGA), and poly(ε-caprolactone) (PCL)) form a hydrophobic micelle core, surrounded by a hydrophilic shell containing the polyplex along with PEG chains to increase dispersibility and provide steric shielding against nucleases.⁴ Within these polyplex micelles, the role of the hydrophobic core is to increase polyplex stability through strong hydrophobic interactions^{4,17,199} and chain crystallization³⁰ while serving as a reservoir for hydrophobic drugs or other agents in combination therapy applications.^{4,22,24,27,29} Although PEG chains in the shell layer of polyplex micelles provide some steric shielding against nucleases, they do not preclude the penetration of nucleases to the nucleic acid cargo over long exposure times¹⁷. One approach has been to design polyplex micelles with hydrophobic domains as physical barriers between the encapsulated nucleic acids and nucleases in the bloodstream.^{8,17,19,28}

### SUMMARY

The present invention relates to polymeric micelle compositions.

In one aspect, the present invention provides a micelle in which a nucleic acid reversibly bound to the polycation-containing block copolymer (“a polyplex”) is embedded within a hydrophobic core, such that each encapsulated nucleic acid is surrounded by a matrix of condensed hydrophobic chains (a “polyplex-in-hydrophobic-core” (PIHC)). In some embodiments, the PIHC micelle includes a contiguous and multichain hydrophobic physical barrier.

In some aspects, the present invention provides a micelle including: a first block copolymer including a polycationic segment and a first hydrophobic segment; and a nucleic acid molecule reversibly bound to the polycationic segment of the block copolymer, and a second block copolymer including a second hydrophobic segment and a water-soluble segment, where the first and second hydrophobic segments form a matrix of condensed hydrophobic chains and form a core together with the nucleic acid molecule; and where the water-soluble segment forms a hydrophilic coronal layer that contains the core.

In some embodiments, the first block copolymer may be poly(ε-caprolactone)-block-poly(2-vinyl pyridine).

In some embodiments, the second block copolymer may be poly(ε-caprolactone)-block-poly(ethylene glycol).

In some embodiments, the micelle may include one or more cores.

In some embodiments, the nucleic acid molecule may be DNA or RNA.

In some aspects, the present invention provides a method for preparing a micelle by: contacting a nucleic acid molecule with a first block copolymer including a polycationic segment and a first hydrophobic segment under conditions suitable for reversible binding to form a polyplex; and contacting the polyplex with a block copolymer including the hydrophobic segment and a water-soluble segment under conditions suitable for condensation of the first and second hydrophobic segments, such that the condensation results in the formation of the micelle.

In some embodiments of the method, the first block copolymer may be poly(ε-caprolactone)-block-poly(2-vinyl pyridine).

In some embodiments of the method, the second block copolymer may be poly(ε-caprolactone)-block-poly(ethylene glycol).

In some embodiments of the method, the micelle may include one or more cores.

In some embodiments of the method, the nucleic acid molecule may be DNA or RNA.

In some aspects, the present invention provides a method of transforming or transfecting a cell, or of delivering a nucleic acid molecule to a cell, by contacting the cell with a micelle as described herein.

In some embodiments of the method, the first block copolymer may be poly(ε-caprolactone)-block-poly(2-vinyl pyridine).

In some embodiments of the method, the second block copolymer may be poly(ε-caprolactone)-block-poly(ethylene glycol).

In some embodiments of the method, the micelle may include one or more cores.

In some embodiments of the method, the nucleic acid molecule may be DNA or RNA.

In some embodiments of the method, the cell may be a prokaryotic cell or a eukaryotic cell.

In some embodiments of the method, the eukaryotic cell may be a tissue or organ.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A is a schematic of a two-step hierarchical self-assembly process to form PIHC micelles in which, in the first step (SA1), pDNA and PCL-b-P2VP undergo electrostatic complexation in a dioxane/acetic acid/water mixture to form the block ionomer micelle intermediate PCL-pDNA.

FIG. 1B is a schematic of a two-step hierarchical self-assembly process to form PIHC micelles in which, in the second step (SA2), PCL-pDNA and PCL-b-PEG undergo microprecipitation via water addition to form PIHC micelles.

FIG. 2 is a graph showing functionalization efficiencies (FE) of PCL-pDNA formed in 70/30 dioxane/acetic acid (v/v) at different water and salt contents. Error bars were determined from triplicate preparations.

FIG. 3 is a graph showing characterization of PCL-pDNA block ionomer micelle intermediate by DLS intensity distributions (CONTIN analysis) and mean effective hydrodynamic diameters, d_{h,eff} (cumulant analysis) of PCL-pDNA in 70/30 dioxane/acetic acid (v/v)+6.2 wt % water (SA1 solvent mixture).

FIG. 4 is a schematic showing the second self-assembly step (SA2) and subsequent workup to produce PIHC micelles by removal of free (unencapsulated) pDNA from dialyzed suspensions by DNase I digestion and centrifugal filtration.

FIG. 5A shows encapsulation efficiencies (EE) of PIHC micelles formed using bulk and microfluidic methods. Error bars were determined from triplicate preparations.

FIG. 5B shows DLS intensity distributions (CONTIN analysis) and mean effective hydrodynamic diameters, d_{h,eff} (cumulant analysis) of bulk (top) and microfluidic (bottom) PIHC micelles in deionized water. For PIHC micelles formed using the microfluidic method, the peak diameters of small and large particle populations in the intensity distribution are 60 and 370 nm, respectively.

FIG. 6 is a graph showing transformation efficiencies versus incubation time for competent E. coli K12 treated with PIHC micelles (closed black circle data points), positive control (open circle data points and dashed linear trendline), and negative control (open circle data points). Positive and negative controls are described in the text.

FIG. 7 is a graph showing size stability data for microfluidic PIHC micelle measured by DLS over two weeks for a single sample. The dashed horizontal line indicates the initial t=1 day measurement. Insignificant deviation from the initial size over 14 days indicates good stability over this time period.

FIG. 8 is a graph showing polydispersity stability data (PDI) for microfluidic PIHC micelle measured by DLS over two weeks for a single sample. The dashed horizontal line indicates the initial t=1 day measurement. Insignificant deviation from the initial polydispersity was observed over the first 12 days.

FIG. 9A is a graph showing cell viability of MDA-MB-231 cells with various dosing levels of PIHC micelles (1-1000 PIHC micelle dose/ppm). Absence of dose dependence over 4 orders of magnitude of dosing levels indicates no cytotoxic effect of the PIHC micelles.

FIG. 9B is a graph showing cell viability of MDA-MB-231 cells with various dosing levels of PIHC micelles (0.1-100 PIHC micelle dose/ppm). Absence of dose dependence over 4 orders of magnitude of dosing levels indicates no cytotoxic effect of the PIHC micelles.

### DETAILED DESCRIPTION

In some embodiments, the present disclosure provides a micelle in which a nucleic acid reversibly bound to the polycation-containing block copolymer (“a polyplex”) is embedded within a hydrophobic core, such that the encapsulated nucleic acid is surrounded by a matrix of condensed hydrophobic chains (a “polyplex-in-hydrophobic-core” (PIHC)). In some embodiments, the PIHC micelle includes a contiguous and multichain hydrophobic physical barrier separating the nucleic acid from the environment.

According, in some embodiments, the present disclosure provides a PIHC micelle including: i) a first block copolymer including a polycationic segment and a first hydrophobic segment, and a nucleic acid molecule reversibly bound to the polycationic segment of the first block copolymer, and ii) a second block copolymer including a second hydrophobic segment and a water-soluble segment, where the hydrophobic segments of the first and second block copolymers form a matrix of condensed hydrophobic chains and form a core together with the nucleic acid molecule; and where the water-soluble segment forms a hydrophilic coronal layer that contains the core. In other words, the core of the PIHC micelle includes the polyplex (the first block copolymer including a polycationic segment and a first hydrophobic segment, and a nucleic acid molecule reversibly bound to the polycationic segment of the first block copolymer, to form a “polyplex-containing core”) as well as the second hydrophobic segment of the second block copolymer, while the coronal layer includes the water-soluble segment of the second block copolymer such that the core and the coronal layer are in different phases i.e., are microphase separated. In some embodiments, the present disclosure provides a single PIHC micelle including one or more polyplex cores.

A “block copolymer” is a copolymer in which chemically distinct monomer units are grouped in discrete blocks along the polymer chain. Block copolymers for use in accordance with the present disclosure may be linear, branched, cyclic, etc. In some embodiments, a block copolymer for use in accordance with the present disclosure may be biocompatible. In some embodiments, a block copolymer for use in accordance with the present disclosure may be suitable for drug delivery.

The first block copolymer as described herein includes a polycationic segment and a first hydrophobic segment. In some embodiments, the first block copolymer may be soluble in organic solvent mixtures, such as polar organic solvent mixtures.

In some embodiments, the first block copolymer may be pH sensitive to allow charging (protonation) of the polycationic segment. In some embodiments, a suitable block copolymer is one in which the polycationic segment is capable of binding to a nucleic acid and the first hydrophobic segment is capable of providing dispersibility in a polar organic solvent followed by condensation with water addition.

In some embodiments, the polycationic segment of the first block copolymer may be an amine-containing polymer which holds positive charges in its protonated form. Accordingly, in some embodiments, the polycationic segment can bind negatively-charged nucleic acids. In alternative embodiments, unbound amine groups of the polycationic segment can act as “proton sponges” to cause osmotic swelling and disruption of the endosomes, thus promoting endosomal escape.

In the first block copolymer, the polycationic segment may, for example, be a poly(2-vinyl pyridine), poly(4-vinylpyridine), poly(ethylene imine), poly(2-(dimethylamino)ethyl methacrylate), polylysine, etc. and the first hydrophobic segment may, for example, be poly(ε-caprolactone), poly(lactic acid), poly(lactic-co-glycolic acid), poly(propylene oxide), polystyrene, etc. or a combination thereof. In some embodiments, the first block copolymer may be poly(ε-caprolactone)-block-poly(2-vinyl pyridine).

In some embodiments, the hydrophobic segment of the first block copolymer may be any polymer that can phase separate from solution upon water addition.

The second block copolymer, as described herein includes a second hydrophobic segment and a water-soluble segment. The hydrophobic segment of the second block copolymer may be any polymer that can phase separate from solution upon water addition and be capable of forming a matrix of condensed hydrophobic chains with the first hydrophobic segment of the first block copolymer, accordingly, the second hydrophobic segment of the second block copolymer may be the same or different from the first hydrophobic segment of the first block copolymer as long the first and second hydrophobic segments are of sufficient compatibility. In some embodiments, the second hydrophobic segment of the second block copolymer is the same as the first hydrophobic segment of the first block copolymer.

In some embodiments, the water-soluble segment of the second block copolymer may be any polymer capable of dispersing the micelles by being solubilized in the solution phase after water addition.

In the second block copolymer, the water-soluble segment may, for example, be a poly(ethylene glycol), polyvinylpyrrolidone, polyvinyl alcohol, poly(acrylic acid), polyacrylamide, polyoxazoline, polyphosphate, etc. In some embodiments, the second block copolymer may be poly(ε-caprolactone)-block-poly(ethylene glycol).

The terms “nucleic acid” or “nucleic acid molecule” encompass both RNA (plus and minus strands) and DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). DNA may include without limitation cDNA, genomic DNA, plasmid DNA, oligonucleotides, antisense and synthetic (e.g., chemically synthesized) DNA, etc. RNA may include without limitation siRNA, mRNA, etc.

In some embodiments, the PIHC micelle may have a hydrodynamic diameter of about 50 nm to about a micrometer, or any value in between, such as 50 nm, 60 nm, 80 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, or 1000 nm. In some embodiments, the PIHC micelle may have a hydrodynamic diameter of a micrometer or less. In some embodiments, the PIHC micelle may have a hydrodynamic diameter of >200 nm. In some embodiments, the PIHC micelle may have a hydrodynamic diameter of >200 nm to about a micrometer. In some embodiments, the PIHC micelle may have a hydrodynamic diameter of about 50 nm to about 200 nm, or any value in between, such as 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm. In some embodiments, the PIHC micelle may have a hydrodynamic diameter of about 60 nm.

In some embodiments, the PIHC micelle may be generally spherical in shape. In some embodiments, the polyplex core may be about 30 nm. In general, the PIHC micelle may be of a size corresponding to the size of the nucleic acid (e.g., genes) i.e. the larger the core, the larger the nucleic acid material that could be incorporated into the micelle, and/or the application (e.g., topical).

In some embodiments, the present disclosure provides a method for preparing a PIHC micelle by contacting a nucleic acid molecule with a first block copolymer comprising a polycationic segment and a first hydrophobic segment under conditions suitable for reversible binding to form a polyplex; and contacting the polyplex with a block copolymer comprising a second hydrophobic segment and a water-soluble segment under conditions suitable for condensation of the first and second hydrophobic segments, where the condensation results in the formation of the micelle.

By “reversible binding,” as used herein, is meant the electrostatic binding of a polymer to a nucleic acid below the pH values where amine groups become protonated/charged and release of the nucleic acid above those pH values.

By “condensation,” as used herein, is meant the coming together of molecules to form a solid phase, above a critical water content, where the hydrophobic blocks phase-separate from solution.

Suitable solvents or mixtures thereof for polyplex formation may be any solvent or mixture that is: sufficiently polar to support charging (protonation) of the polycationic segment; sufficiently non-polar to allow solubilization of the hydrophobic segment; support a pH that permits protonation of the polycationic segment; and/or support the structural integrity of the nucleic acid molecule. It is to be understood that suitable solvents or mixtures thereof for polyplex formation would depend on the choice of segments for the first block copolymer, as described herein. For example, suitable solvents or mixtures thereof may include, without limitation, mixtures of acetic acid with dioxane, dimethylformamide (DMF), tetrahydrofuran (THF), acetone, ethanol, ethylene glycol, depending on the solubility of the segments. Accordingly, in one example, the amount of acetic acid in the mixture would depend upon the pKa of the cationic segment. The amount of acetic acid should be chosen to ensure sufficient charge on the cationic segment for binding but not so much that the nucleic acid is degraded. In some embodiments, a suitable solvent mixture for polyplex formation may include about 20%-40% acetic acid/80%-60% dioxane. In some embodiments, a suitable solvent mixture for polyplex formation may be a 70/30 dioxane/acetic acid (v/v) mixture.

As described herein, the amount of polyplex formation increases with increasing functionalization efficiency (FE) i.e., the higher the FE of the first step, the more nucleic acid is available for encapsulation in the second step. In some embodiments, a suitable solvent or mixture for polyplex formation may be one having a functionalization efficiency (FE) comparable to those in the “no dioxane” case, as described herein, but without significant nucleic acid degradation or loss of functionality. In some embodiments, suitable conditions for polyplex formation have a FE of at least 5%.

Suitable solvents or mixtures thereof for self-assembly of the core and the coronal layer may be any solvent or mixture that can solubilize the second block copolymer.

A suitable amount of water may be determined as described herein, with respect to the critical water content or “cwc.”

It is to be understood that the mixing with water may be performed using any suitable technique known in the art or described herein, for example, injecting water into the solution with rapid mixing; injecting the solution into water into with rapid mixing, microfluidics methods, etc.

Any suitable technique, as described herein or known in the art, such as microprecipitation, dialysis, centrifugal filtration, etc. may be used to obtain the PIHC micelles.

In some embodiments, a PIHC micelle, as described herein, may provide stable encapsulation of nucleic acids while protecting them from constituents in the extracellular environment, for example, deoxyribonucleases (DNases) and ribonucleases (RNases) in the blood. In some embodiments, a PIHC micelle, as described herein, may disperse in the bloodstream and deliver nucleic acids to target cells through, for example, passive or active targeting. In some embodiments, a PIHC micelle, as described herein, may facilitate cellular uptake followed by the release of nucleic acids to, for example, trigger desired transfection events. In some embodiments, the nucleic acid may be released in a slow and/or controlled manner from the PIHC micelle. In some embodiments, a PIHC micelle, as described herein, may be broken down and excreted by a subject, such as a patient, with minimal toxicological effects.

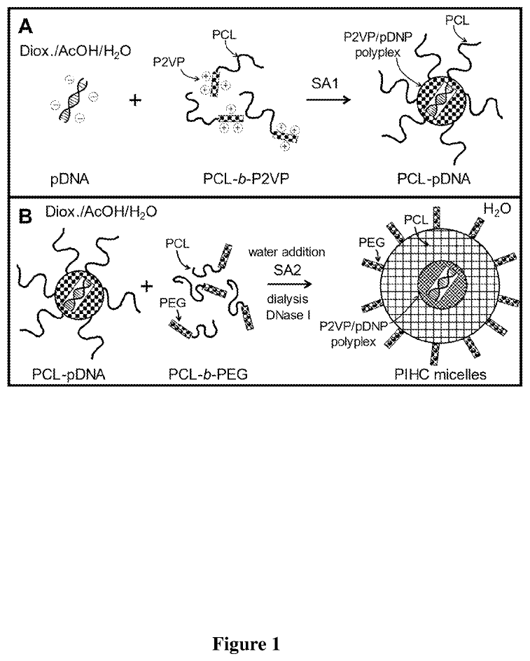

In one example, the present disclosure provides a hierarchical block copolymer self-assembly process to generate a PIHC micelle structure (see, for example, Figures IA-B). PIHC micelles in accordance with the present disclosure are formed in two distinct self-assembly steps: (1) in step 1 (see, for example, SA1, FIG. 1A), nucleic acids are complexed through reversible (e.g., electrostatic) binding with a polycation-containing block copolymer, for example, poly(2-vinyl pyridine) (P2VP) chains of a poly(ε-caprolactone)-block-poly(2-vinyl pyridine) (PCL-b-P2VP) block copolymer, forming stable block ionomer micelles with a polyplex core and a PCL coronal layer (PCL-plasmid DNA (pDNA)); (2) in step 2 (SA2, FIG. 1B), the PCL chains of PCL-pDNA are condensed with the PCL chains of a poly(ε-caprolactone)-block-poly(ethylene glycol) (PCL-b-PEG) block copolymer via water addition and dialysis, forming PIHC micelles. As described herein, we apply this hierarchical approach to form PIHC micelles containing the plasmid DNA (pDNA) pUC18, which codes for antibiotic resistance to ampicillin in bacteria.

In some embodiments, a PIHC micelle as described herein may be used for delivery of a nucleic acid molecule to a cell, for example, a prokaryotic cell, eukaryotic cell, tissue or organ.

In some embodiments, a PIHC micelle as described herein may be used in research, diagnostic, pharmaceutical, veterinary, biomedical, or other applications.

The present invention will be further illustrated in the following examples.

### EXAMPLES

### Experimental Methods

Materials. Poly(ε-caprolactone)-block-poly(ethylene glycol) (PCL(12k)-b-PEG(5k), Ð=1.11, Advanced Polymer Materials Inc.), poly(ε-caprolactone)-block-poly(2-vinyl pyridine) (b-PCL-(35.0k)-b-P2VP(20.5k), Ð=1.10, Polymer Source Inc.), and poly(2-vinylpyrridine) (P2VP(35k), Ð=1.07, Sigma) were used without further purification. All block copolymer solutions were prepared by adding solvent to the solid polymer and stirring overnight before use. Lurai-Bertani (LB) broth (Fisher), glycerol (>99.0%, Sigma), ampicillin (Sigma), trypticase soy agar (TSA, Sigma), hydrochloric acid (HCl, 36.5-38.0%, Sigma), sodium hydroxide solution (1 N, Sigma), ethidium bromide (EtBr, 95%, Sigma), 1,4-dioxane (99.9%+ high-performance liquid chromatography (HPLC) grade, Sigma), acetic acid (99.7%+, Sigma), sodium acetate (99%+, Sigma), pyridine (99.8%, Sigma), sulfuric acid (37% (v/v), Ricca), hydrogen peroxide (30% (w/w), Sigma), ascorbic acid (>99.0%, Sigma), ammonium molybdate tetrahydrate (>99.0%, Sigma), 8-anilino-1-napthalene sulfonic acid ammonium salt (ANSA, >97%, Sigma), sodium sulfite (>98%, Sigma), phosphate standard solution (0.65 mM, Sigma), agarose (Sigma), tris-acetate-ethylenediamine-tetraacetic acid buffer (TAE, Sigma), DNA gel loading dye (Fisher), Dulbecco's modified Eagle's medium (DMEM, Sigma), fetal bovine serum (FBS, Gibco), phosphate-buffered saline (PBS) pH 7.4 (Sigma), trypsin (0.25%, Gibco), phenol red (Fischer), 1′-dioctadecyl-3,3,3′,3′-tetramethylindocarbocyanine perchlorate (DiI, Sigma), 4′,6-diamidino-2-phenylindole (DAPI, Sigma), fluorescein diacetate (95%, Sigma), acetone (>99.5%, Sigma), and DNase I kit: DNase (1 U/μL), DNase I 10× reaction buffer with MgCl₂, and 50 mM ethylenediaminetetraacetic acid (EDTA) (Fisher) were used as received. Super optimal broth with catabolite repression (SOC) medium and calcium chloride manganese buffer (CCMB) medium were made from the ingredients lists below (all purchased from Sigma) and autoclaved prior to use and then stored at room temperature on the benchtop and at 4° C., respectively. The SOC media was composed of tryptone, yeast extract, NaCl (>99.0%) and D-(+)-glucose (>99.5%). The CCMB media was composed of potassium acetate (>99.0%,), glycerol (>99.0%), calcium chloride dihydrate (>99%), manganese chloride tetrahydrate (98%), magnesium chloride hexahydrate (>99.0%), and potassium hydroxide (>85%).

pDNA Isolation and Characterization. Bacterial pUC18 plasmid DNA (pDNA) contains 2686 base pairs and 5372 phosphate groups and has a molecular weight of 1.75×10³ kDa. pDNA was isolated from a frozen stock of E. coli W1130 stored at −80° C. in a 20% (v/v) glycerol/water solution. In a typical isolation process, the frozen stock was streaked onto TSA agar plates containing 200 μg/mL ampicillin and allowed to grow overnight. Ten milliliter LB growth medium was inoculated from the TSA plates and allowed to grow overnight in an incubator at 37° C. with shaking (300 rpm) to produce an overnight culture. Two milliliters of the overnight culture was used to inoculate 2.0 L of LB growth medium and allowed to grow overnight at 37° C. with shaking (300 rpm) to produce an isolation culture, which was divided into 4×500 mL aliquots. pDNA was then isolated from each aliquot into deionized (DI) water using a QIAGEN Plasmid Maxiprep kit, resulting in ˜1 mL of pDNA samples, which were further concentrated to 200-300 μL using centrifugal evaporation. All pDNA samples were characterized using a Thermo Fisher ND-1000 NanoDrop to determine purity and concentration. Since the experiments described in subsequent sections required higher pDNA concentrations than were accessible using a single Maxiprep kit, pDNA stock solutions consisted of multiple (3-6) isolated samples that were pooled and mixed by gentle vortexing. Only isolated samples with an absorbance ratio A_{260:280}=1.8±0.1 were used for pooling. All pooled samples had absorbance ratios of A_{260:280}=1.8±0.1 and A_{260:230}=2.00±0.03 and pDNA concentrations in the range of 700-1000 ng/μL. Finally, all pooled samples were diluted using DI water to a final pDNA concentration of 696 ng/μL and were stored at 4° C. to be used as pDNA stock solutions in the experiments described below.

Fluorescence Characterization of Binding between pDNA and Poly(2-vinylpyrridine). The following fluorescence experiments were carried out to confirm binding between P2VP and pDNA. Inside a quartz fluorescence cuvette, 45 μg of pDNA (64.5 μL of pDNA stock) were suspended in 3.0 mL of DI water that had been previously acidified to pH 3 using HCl. Then, aqueous EtBr solution (10.0 mg/mL) was added to the cuvette at a molar ratio of 1:1 EtBr/phosphate (5.5 μL), and the contents of the cuvette were mixed on an orbital shaker at 300 rpm for 30 s, followed by a fluorescence measurement of the mixture. Next, P2VP homopolymer was dissolved in acidified DI water (pH 3, HCl) to a concentration of 17.0 mg/mL. The P2VP solution was then added to the cuvette at a molar ratio of 4:1 pyridinium/phosphate (3.4 μL) and the contents of the cuvette were mixed at 300 rpm for 12 h, followed by a second fluorescence measurement of the mixture. Finally, 60 μL of 1 N NaOH were added and the contents of the cuvette were mixed at 300 rpm for 1 h, followed by a final fluorescence measurement of the neutralized mixture (pH ˜7). All fluorescence measurements were carried out on an Edinburgh Instruments FLS920 fluorimeter, with excitation at 285 nm from a Xe900 xenon are lamp and emission measured by a R9289 photomultiplier tube. Fluorescence emission spectra were collected between 565 and 800 nm and corrected for instrument response.

Functionalization of pDNA with PCL-b-P2VP (SA1). For each experiment, 45.0 μg of pDNA (64.5 μL of pDNA stock) were transferred to a 1.5 mL Eppendorf tube followed by dilution with variable quantities of DI water (0, 17, 57, 195, or 340 μL). The resulting pDNA solutions were added with stirring to 500-600 μL of a 70/30 (v/v) mixture of dioxane/acetic acid in a Teflon centrifuge tube. Next, more 70/30 (v/v) dioxane/acetic acid mixture was added to bring the volume of the tube to 1.0 mL, such that the pDNA concentration was 45 ng/μL. The solutions were weighed to determine the following variable water concentrations: 6.2, 7.8, 11.6, 24.9, and 39.1 wt %. For functionalization in the presence of salt, sodium acetate was dissolved in the 70/30 (v/v) mixture of dioxane/acetic acid such that the salt concentration after the addition of the aqueous pDNA solution was either 0.01 or 0.1 M. Finally, PCL-b-P2VP in dioxane (17.0 mg/mL) was added to the pDNA solution at a molar ratio of 4:1 pyridinium/phosphate (9.3 μL). The resulting SA1 mixture was stirred at 800 rpm overnight at 21° C.

Determination of pDNA Functionalization Efficiency. To quantify the efficiency of pDNA functionalization, the following procedure was applied after the overnight stirring of the SA1 mixture. First, 2.0 mL of chilled isopropanol (stored at −4° C.) was added to the centrifuge tube containing the SA1 mixture followed by incubation at −4° C. for 4 h. Next, the mixture was centrifuged at 12 000 g for 30 min to yield a colorless opaque pellet containing a blend of functionalized and unfunctionalized pDNA. This pellet was isolated by decanting off the clear liquid and then dissolving in 3.0 mL of chloroform, followed by three wash steps using 3.0 mL of acidified DI water (pH 3, acetic acid) for each step. After each wash step, organic and aqueous layers were separated in a separatory funnel and the bottom organic layer was collected for the next wash. The chloroform solution was transferred to a test tube and the solvent was removed by gently passing air over the test tubes for 24 h. After drying, the phosphate content in the chloroform solution was determined using a phosphate assay and compared to the phosphate content in the total amount of 45 μg of pDNA used in the SA1 experiment. Since only functionalized pDNA (PCL-pDNA) is soluble in chloroform, the following equation was used to determine the functionalization efficiency of pDNA (FE)

$$F E = \frac{{[ phosphate ]}_{chloroform}}{{[ phosphate ]}_{total pDNA}} \times 100 \% .$$

In the above equation, phosphate concentrations represent the number of moles of phosphate extracted into 200 μL of DI water.

Reported FE values were determined by averaging the values from triplicate preparations under the same conditions (water and salt content). Standard errors (±σ) on FE values were calculated from the standard deviation (s) of triplicate values, σ=s/√3. Optimized functionalization (highest FE values) was found at 6.2 wt % water and no salt, such that these optimized conditions were used for all experiments described below.

Release of pDNA from PCL-pDNA. To confirm the structural integrity of pDNA following the SA1 step, pDNA was released from the copolymer for gel electrophoresis analysis in the following manner. First, after the overnight stirring of the SA1 mixture (6.2 wt % water and no salt added), a pellet containing functionalized and unfunctionalized pDNA was isolated and dissolved in chloroform as described in the previous section. Then, after washing 3× with acidified water (pH 3, acetic acid) to remove unfunctionalized pDNA, the chloroform fraction containing PCL-pDNA was transferred from the separatory funnel to a new vial. Next, 3.0 mL of DI water was layered on top of the chloroform and 60 μL of pyridine was added to the chloroform layer. The two-phase mixture was stirred at 800 rpm for 3 h at 21° C. followed by separation of chloroform and aqueous layers in a separatory funnel. The aqueous fraction containing released pDNA was concentrated by centrifugal evaporation to a volume of <100 μL and then analyzed by gel electrophoresis.

Bulk Encapsulation of PCL-pDNA in PIHC Micelles (Bulk SA2). After overnight stirring of the optimized SA1 mixture (6.2 wt % water and no salt), PCL-b-PEG in dioxane (10.4 mg/mL) was added at a mass ratio of 1:10 PCL-b-PEG/pDNA+PCL-b-P2VP (2.0 μL). The solution was allowed to stir at 800 rpm for 3 h and then transferred to a small clean vial. To initiate self-assembly, water was added to the solution at a constant injection rate of 120 μl/min using a syringe pump with stirring at 800 rpm until a water content of 33 wt % was reached. Then, 10 mL of DI water was added, followed by immediate vortex mixing. Finally, the dispersion was transferred to 6-8 kDa molecular weight cut-off (MWCO) dialysis tubing and dialyzed against DI water for 18 h, changing the water every hour for the first 4h.

After dialysis, unencapsulated pDNA was removed from the aqueous dispersion using a combination of enzymatic digestion and centrifuge filtration. First, the dialyzed sample was concentrated to 2.5 mL using a centrifuge filter (Macrosep, PALL) with MWCO=100 kDA by centrifuging for 12 min at 5000 g. The concentrated sample was then split equally into two 1.5 mL microcentrifuge tubes. To each tube, 150 μL of DNase I 10× reaction buffer was added, followed by the addition of water to the 1.5 mL mark and gentle vortexing. Then, 12.5 μL of DNase I enzyme was added to each tube and the mixture was again vortexed gently, followed by 1 h incubation at 21° C. After incubation, 22.5 μL of EDTA solution (50 mM) was added to each tube and the mixture was vortexed gently. Finally, the sample was recombined and washed by adding 12.5 mL of DI water then concentrating to 2.5 mL using a centrifuge filter (Macrosep, PALL) with MWCO=100 kDA by centrifuging for 12 min at 5000 g. The wash step was repeated two more times. The particles after removal of unencapsulated pDNA were designated PIHC micelles (bulk preparation).

Microfluidic Reactor Fabrication. The microfabrication steps followed previously described procedures.⁴² First, negative masters were fabricated on silicon wafers (Silicon Materials) using the negative photoresist SU-8 100 (Microchem). A 150 μm thick SU-8 film was spin-coated at 2000 rpm onto the silicon wafer and heated at 65° C. for 12 min and then at 95° C. for 50 min. After the wafer was cooled, a photomask was placed directly above, and the wafer was exposed to UV light for 100 s. Then, the UV-treated film was heated at 65° C. for 1 min and then 95° C. for 20 min. Finally, the silicon wafer was submerged in SU-8 developer (Microchem) and rinsed with isopropanol until all unexposed photoresist was removed. Microfluidic chips were fabricated from poly(dimethylsiloxane) (PDMS) using a SYLGARD 184 silicon elastomer kit (DowCorning). For fabrication of all PDMS chips, the elastomer and curing agent were mixed at a 7:1 ratio and degassed under vacuum. The resulting mixture was poured over a clean negative master chip in a Petri dish and further degassed until all remaining air bubbles were removed. The PDMS was heated at 85° C. until cured (˜20 min) and then peeled from the negative master; holes were punched through the reservoirs of the resulting PDMS chip to allow for the insertion of tubing. A thin PDMS film (substrate layer) was also made on a glass slide by spin-coating a 20:1 elastomer/curing agent mixture followed by curing. The substrate layer was then permanently bonded to the base of the microfluidic reactor (channel layer) after both components were exposed to oxygen plasma for 90 s. The resulting reactor has a set channel depth of 150 μm and consists of a sinusoidal mixing channel 100 μm wide and a straight processing channel 200 μm wide.

Flow Delivery and Control. The steps of flow delivery and control followed previously described procedures.⁴² Pressure-driven flow of liquids to the reactor inlet was provided using 1 mL gastight syringes (Hamilton) mounted on syringe pumps (Harvard Apparatus). The microfluidic chip was connected to the liquid syringes via 1/16th in. (OD) Teflon tubing (Mandel Scientific). Argon (Ar) gas flow was introduced to the chip via an Ar tank regulator and a downstream regulator (Johnston Controls) for fine adjustments. The chip was connected to the downstream regulator through a 1/16th in. (OD)/100 μm (ID) Teflon tube (Mandel Scientific). The liquid flow rate (Q_{liq}) was programmed via the syringe pumps, and the gas flow rate (Q_{gas}) was fine-tuned via the downstream pressure regulator to set a total nominal flow rate (Q) of 200 μL/min. Due to the compressible nature of the gas and the high gas/liquid interfacial tension, discrepancies arise between the nominal (programmed) and actual values of Q_{gas}, Q_{gas}/Q_{liq}, and the total flow rate (Qₜₒₜₐₗ). Therefore, actual values of Q_{gas}, Q_{gas}/Q_{liq}, and Qₜₒₜₐₗ=Q_{gas}+Q_{liq} for each microfluidic experiment were calculated from the average volume of gas bubbles in the microchannels. Specifically, an image of microchannels was captured using a Genie Nano-C1280 camera (1stVision) equipped with an On-Semi Python1300 sensor and a C-Mount Manual Iris Varifocal lens (1/1.8 in., 4-13 mm, f/1.5) (Tamron) at each of three different time periods at the beginning, middle, and end of the sample collection process. Analysis of the gas bubbles and liquid plugs within the microfluidic reactor was achieved using image analysis software (ImageJ), which gives the end-to-end distance of individual gas bubbles and liquid plugs, L_{gas,i} and L_{liq,i}, respectively, under a given set of flow conditions. The gas-to-liquid flow ratio, Q_{gas}/Q_{liq}, was determined from each image as the ratio between measured ΣᵢL_{gas,i} and ΣᵢL_{liq,i} (i=20-50). Actual gas-to-liquid flow ratios for all experimental runs are reported as average values determined from three images for each run. All actual Qₜₒₜₐₗ values were within 10% of nominal Q values.

Microfluidic Encapsulation of PCL-pDNA in PIHC Micelles (Microfluidic SA2). After overnight stirring of the optimized SA1 mixture (6.2 wt % water and no salt), PCL-b-PEG in dioxane (10.4 mg/mL) was added at a mass ratio of 1:10 PCL-b-PEG/pDNA+PCL-b-P2VP (2.0 μL). The solution was allowed to stir at 800 rpm for 3 h and was then used as the PCL-pDNA/PCL-b-PEG stream for microfluidic assembly. For microfluidic preparation of PIHC micelles, the following three liquid streams were combined to give stable gas-segmented liquid plugs within the reactor: (1) the PCL-pDNA/PCL-b-PEG stream, (2) pure dioxane, and (3) pure DI water. The flow rates of the three liquid streams were equal for all runs, and the steady-state on-chip water concentration was 33 wt %. Due to dilution by the additional dioxane stream, PCL-pDNA and PCL-b-PEG were 2× more dilute during water addition in microfluidic channels than during bulk water addition. For each microfluidic run, 3×1 mL of liquid portions were collected from the chip into vials containing 10 mL of DI water and then combined. To remove residual dioxane and acetic acid, the resulting PNP dispersions were then immediately transferred into a 6-8 kDa MWCO dialysis membrane and dialyzed against DI water for 18 h, with changing of DI water every hour for the first 4 h. After dialysis, unencapsulated pDNA was removed from the aqueous dispersion using a combination of enzymatic digestion and centrifuge filtration as described in the bulk SA2 section. The final particles after removal of unencapsulated pDNA were designated PIHC micelles (microfluidic preparation).

Preparation of Positive and Negative Control Samples. The positive control sample was prepared using an equivalent amount of pDNA, which was exposed to the same solvents, dialysis, and centrifugal filtration as PIHC micelle formation. However, no copolymers were added and no DNase I digestion step was applied to the positive control sample. Briefly, 45.0 μg of pDNA (64.5 μL of pDNA stock) was added to a 30% (v/v) mixture of dioxane/acetic acid in a Teflon centrifuge tube such that the total volume was 1.0 mL. The solution was then injected into 10 mL of DI water at 120 μL/min followed by overnight dialysis for 18 h, changing the water every hour for the first 4 h. Following dialysis, the aqueous pDNA dispersion was concentrated to 2.5 mL by centrifugal filtration, then washed by adding 12.5 mL of DI water, and reconcentrating to 2.5 mL. The wash step was repeated two more times, resulting in a final aqueous dispersion of 2.5 mL, which was designated the positive control sample. The mass of pDNA in the positive control was determined to be 41 μg using the phosphate assay, indicating that <10% pDNA was lost in the various steps described above. The negative control sample was prepared using equivalent amounts of pDNA and copolymers and with the same solvents, dialysis, and centrifugal filtration steps as PIHC micelle formation. However, the pDNA was added after the SA2 step in the preparation of the negative control sample such that it was not encapsulated during subsequent DNAase digestion. Briefly, 9.2 μL of PCL-b-P2VP in dioxane (17.0 mg/mL) and 2.0 μL of PCL-b-PEG in dioxane (10.4 mg/mL) were added to a 30% (v/v) mixture of dioxane/acetic acid such that the total volume was 1.0 mL. The solution was then injected into 10 mL of DI water at 120 μL/min followed by overnight dialysis for 18 h, changing the water every hour for the first 4 h. After dialysis, 45.0 μg of pDNA (64.5 μL of pDNA stock) was added to the resulting dispersion, followed by concentrating the dispersion to 2.5 mL using centrifugal filtration. The concentrated sample was then split equally into two 1.5 mL microcentrifuge tubes, and to each tube 150 μL of DNase I 10× reaction buffer was added. After the addition of water to 1.5 mL, the mixture was vortexed, followed by the addition of 12.5 μL of DNase I enzyme to each tube. The mixture was vortexed again, then incubated for 1 h at 21° C. Next, 22.5 μL of EDTA solution (50 mM) was added to each tube. After vortexing, the sample was recombined and washed by adding 12.5 mL of DI water, then concentrated to 2.5 mL using centrifuge filtration. The wash step was repeated two more times, resulting in a final aqueous dispersion of 2.5 mL, which was designated the negative control sample.

Determination of pDNA Encapsulation Efficiency. To determine pDNA encapsulation efficiencies, the pDNA content in bulk and microfluidic PIHC micelle samples, and in the positive and negative controls, were analyzed. First, the aqueous dispersion (2.5 mL) was transferred to a test tube and water was removed by gently passing air over the test tube for 24 h. After drying, the pDNA content was determined using a phosphate assay. For PIHC micelles, all unencapsulated pDNA had been removed by digestion with DNase land subsequent washing steps, such that the remaining pDNA was assumed to be encapsulated within the micelles. Therefore, the following equation was used to determine the encapsulation efficiency (EE) of pDNA

$$E E = \frac{{[ phosphate ]}_{sample}}{{[ phosphate ]}_{total pDNA}} \times 100 \% .$$

In the above equation, [phosphate]ₛₐₘₚₗₑ represents the number of moles of phosphate extracted from the dried PIHC micelle sample or control into 200 μL of DI water and [phosphate]_{total pDNA} represents the concentration of phosphate in the total starting mass of pDNA (45 μg) extracted into 200 μL of DI water (0.69 mM). Reported EE values for bulk and microfluidic PIHC micelle preparations were determined by averaging the values from triplicate preparations under the same conditions. Standard errors (±σ) on EE values were calculated from the standard deviation (s) of triplicate values, σ=s/√3. For the positive control sample described in the previous section, the EE value was determined to be 91%, confirming that most of the initial pDNA could be recovered and measured without DNase I exposure, with <10% lost in the centrifugal filtration and washing process.

Release of pDNA from PIHC Micelles. To confirm the structural integrity of encapsulated pDNA following SA1, SA2, and DNase I digestion of unencapsulated pDNA, encapsulated pDNA was released from microfluidic PIHC micelles for gel electophoresis analysis. First, the aqueous micelle dispersion (2.5 mL) was concentrated by centrifuge evaporation to a volume of <200 μL. The resulting concentrated dispersion was then added to 100× excess (v/v) of acetonitrile to dissociate the PIHC micelles. A white precipitate was observed, attributed to salts from DNase I buffers. The acetonitrile was carefully decanted from the vial containing the salts and transferred to a centrifuge tube. The acetonitrile solution containing free PCL-b-PEG chains and PCL-pDNA was chilled for 4 h at −4° C. in the freezer and then centrifuged at 12 000 g for 30 min to pellet the PCL-pDNA fraction. The supernatant was discarded, and the pellet was dissolved in 3 mL of chloroform. Next, 3.0 mL of DI water was layered on top of the chloroform and 60 μL of pyridine was added to the chloroform layer. The two-phase mixture was stirred at 800 rpm for 3 h at 21° C. followed by separation of chloroform and aqueous layers in a separatory funnel. The aqueous fraction containing released pDNA was concentrated by centrifugal evaporation to a volume of <20 μL and then analyzed by gel electrophoresis.

Bacterial Cell Transformation Experiments. To prepare competent E. coli K12 cells for PIHC micelle and control transformation experiments, 10 mL of sterile LB broth was inoculated with E. coli K12 and allowed to grow overnight at 37° C. with shaking to produce an overnight culture. Then, 50.0 mL of sterile LB broth was inoculated with 200 μL of the overnight culture and allowed to grow to OD600=0.5. Cells were collected by centrifugation at 1000 g for 14 min, resuspended in 17.0 mL of calcium chloride manganese buffer (CCMB), and then collected again by centrifugation at 1000 g for 10 min. Finally, the cells were resuspended in 4.16 mL of CCMB buffer and stored overnight at −80° C.

Transformation experiments were carried out in triplicate using a single sample of PIHC micelles prepared using microfluidic encapsulation along with the positive and negative control samples described above. First, three empty 13 mL culture tubes (for PIHC micelles, positive control, and negative control) were chilled on ice for at least 30 min, while the competent cells were thawed on ice. Then, 5.0 μL of the PIHC micelle sample, positive control sample, and negative control sample were added to each of the culture tubes, followed by the addition of 200 μL of competent cells to each tube. All tubes were incubated on ice for 25 min, then transferred to 42° C. water for 70 s, and then back to ice for 2 min. Next, 1 mL of SOC medium was added to each tube and the tubes were incubated at 37° C. with 300 rpm shaking. At incubation times of 1, 3, and 8 h, 100 μL from each tube was plated on agar Petri dishes containing 200 μg/mL ampicillin and allowed to grow overnight at 37° C.

To determine transformation efficiencies (CFU/μg pDNA), the number of colonies on each plate were counted and divided by the pDNA masses (in μg) used in the transformation based on pDNA masses in the sample and controls. Reported transformation efficiency values were determined by averaging the values from triplicate transformation experiments. Standard errors (to) on transformation efficiency values were calculated from the standard deviation (s) of triplicate values σ=s/√3. The masses of pDNA in the negative and positive control samples were determined to be 0 and 41 μg using the phosphate assay. The mass of pDNA in the PIHC micelle sample was determined to be 2.9 μg based on the initial mass of 45 μg and the average EE value for microfluidic encapsulation (6.5±1.1%).

Fluorescence Imaging of PIHC Micelle Uptake in MDA-MB-231 Cells. To confirm the uptake of PIHC micelles into cells using fluorescence microscopy, microfluidic PIHC micelles were prepared as previously described but with the following two additional steps for labeling pDNA and PCL components with different fluorescent dyes. First, before the SA1 step, 45 μg (64.5 μL of pDNA stock) was transferred to a 1.5 mL Eppendorf tube and 2.0 μL of DAPI solution (1.0 mg/mL in DI water) was added, followed by overnight incubation in the dark at 4° C. The SA1 experiment was then conducted as described previously. Second, before the microfluidic SA2 step, 4.0 μL of a DiI solution (0.1 mg/mL in dioxane) was added immediately after the addition of PCL-b-PEG to the SA1 mixture. The SA2 experiment was then conducted as described previously to produce DAPI/DiI-labeled PIHC micelles. DAPI/DiI-labeled PIHC micelles were imaged by depositing a large drop of aqueous suspension onto a microscope slide, then sealing a coverslip on top of the drop with nail polish and allowing the slide to dry overnight. Fluorescence images were captured using a ZEISS LSM 980 with Airyscan 2. The excitation and emission wavelengths were 365 and 460 nm, respectively, for the blue channel and 585 and 650 nm, respectively, for the red channel.

Human breast cancer cells were used as a model system to determine the cell uptake of PIHC micelles. MDA-MB-231 cells were grown to ˜70% confluence in 75 cm² tissue culture flasks, then trypsinized, collected, and pelleted by centrifugation (5 min at 313 g). The cell pellet was then suspended in DMEM, and the cell concentration was determined using a hemocytometer. After the initial cell concentration was determined, the suspension was diluted to 1.0×10⁵ cells/mL. Next, a pipette was used to seed the cells at a density of 3.0×10⁵ cells per well in a six-well plate (Costar), and the plate was then incubated for 24 h at 37° C. with 5% CO₂ for cell adhesion. Within each well, aqueous dispersions of DAPI/DiI-labeled PIHC micelles were mixed 1:5 (v/v) with the cell media for a final PIHC micelle concentration of 0.014 mg/mL. After 4 h incubation at 37° C., the cells were washed 3× with 1 mL of PBS buffer. Fluorescein diacetate was dissolved to a concentration 0.05 mg/mL in a solution of 1.0 wt % acetone in DI water. Then, 1 mL of the fluorescein diacetate solution was added to each well, and the cells were incubated in the dark for 5 min, then washed 3× with 1 mL of PBS and immediately imaged. Fluorescence images were captured using a Cytation5 microplate reader equipped with a fluorescent microscope (BioTek), with three fluorescent filter cubes of emission (nm)/excitation (nm)=377/447, 469/525, and 586/647. Images were taken using a 20× objective.

Cytotoxicity Experiments. MDA-MB-231 breast cancer cells were grown to ˜80% confluence in Dulbecco's modified Eagle's medium (DMEM) supplemented with 10% fetal bovine serum (FBS), 1% penicillin, and 1% streptomycin in a 75 cm² tissue culture flask and maintained at 37° C. with 5% CO₂ in a tissue culture incubator. Cells were then trypsinized, collected, and pelleted by centrifugation at 4° C. and 1200 rpm for 5 min. The cell pellet was then resuspended in DMEM media, and the cell concentration was determined using a hemocytometer. After the initial cell concentration was determined, the suspension was diluted to 1.0×10⁵ cells/mL. Next, a multichannel pipet was used to fill a 96-well plate with 100 μL/well of the diluted cell suspension. The cell-loaded plates were then incubated for 24 h at 37° C. under an atmosphere of 5% CO₂. After a 24 h cell incubation, 6.5 μL of aliquots of PIHC dispersions were diluted to a total volume of 650 μL using DMEM media. Serial dilutions were carried out, and then 100 μL of each diluted stock was added to the appropriate well of the 96-well plate (containing ˜1.0×10⁴ cells in 100 μL of media, as described above), to generate a range of different concentrations for analysis. The treated cells were incubated for 48 h at 37° C. under a 5% CO₂ atmosphere. To determine cell viability, 20 μL of CellTiter-Blue was added to each well after 48 h had passed. After the addition of the CellTiter-Blue, the 96-well plates were incubated for 4 h (5% CO_{2, 37}° C.), and then fluorescence readings were recorded on a 96-well plate reader (λₑₓ=560 nm; λₑₘ=590 nm emission). Cell viability was calculated for each well based upon the following formula

$$cell viability \% = [ \frac{( {S}_{0} - {B}_{0} )}{( {B}_{t} - {B}_{0} )} ] \times 100$$

where S is the sample reading (cells+PIHC micelles+media), Bₜ is the average reading for the untreated population of cells (cells+media), and B₀ is the blank (media only).

Gel Electrophoresis. Samples were mixed in a 1:6 (v/v) ratio with DNA gel loading dye, and then 18.0 μL of each solution was subjected to electrophoresis (100 mV for 1 h) in a 1% agarose gel in 1×TAE buffer. Gels were stained by immersion in an aqueous EtBr bath (0.5 μg/mL) for 15 min and then imaged in an AlphaImager Gel Imaging System (Alpha Innotech). All gel runs were compared against a 1 kb DNA ladder (Fisher).

Phosphate Assays. Phosphate assays enabled quantitative determination of pDNA content in multicomponent samples. To prepare a sample for a phosphate assay, the liquid sample was transferred to a test tube, and then the solvent was removed by passing air over the test tube for 24 h. Next, 200 μL of DI water was added to the dried sample. In separate test tubes, 200 μL of phosphate standard solutions were prepared with known concentrations of 0, 0.15, 0.30, 0.45, and 0.65 mM phosphate. Next, 400 μL of sulfuric acid was added to each test tube, followed by vortex mixing. Then, 200 μL of hydrogen peroxide was added to each test tube, followed by immediate vortexing and capping each test tube with a glass marble. (Warning: piranha solution is a powerful oxidizer and must be handled with extreme care!!) The tubes were heated to between 205 and 215° C. and maintained within this temperature range for 90 min and then cooled at room temperature for 5 min. Next, 100 μL of aqueous ascorbic acid solution (10 wt %), 4.6 mL of ammonium molybdonate tetrahydrate solution (0.890 mM) in 0.25 N sulfuric acid, and 200 μL of 0.209 mM ANSA solution in 14.3 wt % aqueous sodium sulfite were added sequentially to each test tube, vortexing immediately after addition of each solution. Finally, the test tubes were heated at 100° C. for 15 min followed by colorimetric measurements. A standard calibration curve was generated from absorbance values at 830 nm on an Agilent 8453 UV-vis spectrophotometer, and unknown phosphate concentrations were determined by comparison with the calibration curve.⁵²

Critical Water Content Determination. Static light scattering measurements were carried out to determine the cwc of PCL-b-P2VP under conditions of pDNA functionalization and also the cwc of PCL-b-PEG under conditions of microfluidic PIHC micelle formation. Light scattering experiments were performed on a Brookhaven Instruments photocorrelation spectrometer equipped with a BI-200SM goniometer and a BI-Mini-L30 30 mW red (636 nm) compact diode laser, at a scattering angle of 900 and a temperature of 25° C.

To prepare the PCL-b-P2VP sample solution, a 1.0 mL of mixture of 45 μg of pDNA and 158 μg of PCL-b-P2VP in 70/30 (v/v) dioxane/acetic acid was prepared (6.2 wt % water and no salt) as described previously (SA1 step), except that the mixture was prepared in a clean scintillation vial and the dioxane/acetic acid mixture was prefiltered to remove dust (2×0.20 μm² nominal pore size Teflon syringe filters, VWR). After overnight stirring of the SA1 mixture, prefiltered DI water (2×0.20 μm² nominal pore size nylon syringe filters, National Scientific) was added in successive 0.03-0.06 g quantities using a micropipette. After each addition of water, the sample was allowed to equilibrate for 20 min before the scattered light intensity was measured. The resulting scattered light intensity was plotted vs water concentration. In an attempt to obtain light scattering measurements for water contents below 6.2 wt %, an equivalent amount of pDNA was also added from a more concentrated stock (790 ng/mL), such that the initial water content was 5.7 wt %; at that water content, pDNA was observed by eye to precipitate in the dioxane/acetic acid mixture before the P2VP-b-PEG copolymer was added.

To prepare the PCL-b-PEG sample solution, 4.0 mL of dioxane was added to 4.0 mL of 70/30 (v/v) dioxane/acetic acid in a clean scintillation vial, followed by the addition of 8.0 μL of 10.4 mg/mL PCL-b-PEG in dioxane. To remove dust, dioxane and the dioxane/acetic acid mixture were prefiltered (2×0.20 μm² nominal pore size Teflon syringe filters, VWR) and the 10.4 mg/mL PCL-b-PEG solution was also prefiltered (2×0.45 μm² nominal pore size Teflon syringe filters, VWR). After 20 min equilibration, filtered DI water (2×0.20 μm² nominal pore size nylon syringe filters, National Scientific) was added in successive 0.03-0.06 g quantities. After each addition of water, the sample was allowed to equilibrate for 20 min before the scattered light intensity was measured. The resulting scattered light intensity was plotted vs. water concentration.

Dynamic Light Scattering and ζ Potential. DLS measurements were carried out for determination of hydrodynamic sizes and size distributions following SA1 and SA2 self-assembly, and ζ potential measurements were carried out following SA2 self-assembly on a Brookhaven Instruments ZetaPALS Analyzer equipped with a solid-state Laser (660 nm) with a maximum power output of 35 mW. All DLS measurements were performed at an experimental temperature of 25° C. and at a scattering angle of 90°. Solvents used for DLS sample preparation were prefiltered using 2×0.20 μm² nominal pore size nylon syringe filters (National Scientific) for DI water and 2×0.20 μm² nominal pore size Teflon syringe filters (VWR) for dioxane/acetic acid.

For DLS of PCL-pDNA following the SA1 step, the following sample preparation steps were carried out after overnight stirring of the SA1 mixture. First, 2.0 mL of chilled isopropanol (stored at −4° C.) was added to the mixture, followed by incubation at −4° C. for 4h. Next, the mixture was centrifuged at 12 000 g for 30 min to yield a colorless opaque pellet that was isolated by decanting off the clear liquid. The pellet was then resuspended in 1.0 mL of prefiltered SA1 solvent mixture (30% (v/v) dioxane/acetic acid with 6.2 wt % water) by gentle vortexing and transferred to a dust-free cuvette for DLS measurements. For DLS of PIHC micelles following the SA2 step and DNase I digestion and washing, 200 μL of the micelle dispersion was added to 3.0 mL of prefiltered water in a dust-free cuvette for DLS measurements.

The determination of PCL-pDNA hydrodynamic size in the SA1 solvent mixture required calculations of the viscosity (η=1.18 cP) and refractive index (n=1.39) of the SA1 solvent mixture at 25° C. The viscosity of the mixture was calculated using the Refutas equation⁵³

$$η = exp ( exp ( \frac{( {\sum }_{i = 0}^{N} {w}_{i} \times ( 14.534 \times ( ln ( {η}_{i} + 0.8 ) ) + 10.975 ) ) - 10.975}{14.534} ) ) - 0.8$$

where η_{D}=1.22 cP, η_{A}=1.15 cP, and η_{W}=0.91 cP are the viscosities of dioxane, acetic acid, and water components, respectively. In the SA1 mixture, the mass fractions of the components are w_{D}=0.63, w_{A}=0.31, and w_{W}=0.07. The refractive index of the mixture was calculated using a weighted sum (by volume fractions) of refractive index values for the liquid components⁵¹

$$n = {\sum }_{i = 1}^{N} ( {ϕ}_{i} \times {n}_{i} )$$

where n_{D}=1.42, n_{A}=1.37, and n_{W}=1.33 are the refractive indexes of dioxane, acetic acid, and water components, respectively. In the SA1 mixture, the volume fractions of the components are ϕ_{D}=0.65, ϕ_{A}=0.28, and ϕ_{W}=0.06.

Reported d_{h,eff} values were determined using the method of cumulants by averaging values from triplicate preparations under the same conditions. Standard errors (±σ) on d_{h,eff} values were calculated from the standard deviation (s) of triplicate values σ=s/√3.

Transmission Electron Microscopy. Negatively stained samples for TEM imaging were prepared by depositing a drop of ˜0.1 mg/mL PIHC micelle dispersion on a Formvar/carbon-coated 200 mesh copper TEM grid (Ted Pella Inc.), followed by a drop of 1 wt % uranyl acetate aqueous solution as a negative staining agent. Excess liquid was immediately removed using lens paper, followed by drying of the remaining liquid under ambient conditions. Imaging was performed on a JEOL JEM-1400 transmission electron microscope, operating at an accelerating voltage of 80 kV and equipped with a Gatan Orius SC1000 CCD camera.

RESULTS

Reversible Binding of pDNA with P2VP Homopolymer. PCL-b-P2VP was chosen as the polycation-containing block copolymer component. The plasmid pUC18 was chosen for as a proof-of-concept based on its relatively small size and extensive study in the literature.³¹⁻³⁴ However, since the use of P2VP as a nucleic acid-binding polycation had not been widely explored,³⁵ we were interested in confirming reversible polyplex binding in a model system of pUC18 and P2VP homopolymer in acidic aqueous media.

Ethidium bromide (EtBr) was used as a fluorescent probe in a competitive binding assay. EtBr is known to intercalate between base pairs in the DNA double helix, which significantly increases its fluorescence intensity; therefore, binding of DNA with polycations can be monitored via displacement of EtBr and concomitant decrease in EtBr fluorescence.³⁶ Following an initial fluorescence measurement of the EtBr-pDNA complex at pH=3 (acetic media), the EtBr emission intensity decreased significantly after the addition of poly(2-vinyl pyridine) (P2VP) at a ratio of 4:1 pyridinium/phosphate, indicating the displacement of EtBr as negatively charged pDNA formed a polyplex with positively charged P2VP. Then, when the pH was increased by sodium hydroxide (NaOH) addition, deprotonation of pyridinium groups led to the release of bound pDNA from the polymer back into the solution, allowing the EtBr-pDNA complex to reform with an increase in EtBr fluorescence. These results indicated that P2VP blocks of PCL-b-P2VP can form reversible polyplexes with pDNA.

Optimization of Solvent Conditions for PCL-pDNA Formation (SA1 Step). We next investigated solvent conditions for the formation of a polyplex between pDNA and the P2VP blocks of PCL-b-P2VP (SA1, FIG. 1A) by evaluating the SA1 process at a constant ratio of 4:1 P2VP pyridinium/pDNA phosphate in various mixtures of dioxane, acetic acid, water, and salt.

Upon the formation of the desired block ionomer micelle polyplexes (SA1, FIGS. 1A-B), the complexed pDNA becomes functionalized by the surrounding coronal layer of PCL chains, rendering it soluble in nonpolar organic solvents such as chloroform. Therefore, we were able to characterize the efficiency of pDNA functionalization in the following manner. First, various SA1 mixtures were pelleted down by the addition of chilled isopropanol followed by centrifugation; the resulting pellet was then extracted into chloroform and the chloroform solution was washed several times with acidified water (pH=3) to remove any unfunctionalized pDNA. Finally, the amount of pDNA in the chloroform layer was determined using a phosphate assay and compared to the total initial pDNA amount (45 μg). The resulting percentage was taken to be the functionalization efficiency (FE). In preliminary experiments, we carried out the SA1 process at extreme dioxane/acetic acid ratios of 0/100 and 99/1 (v/v). We found that FE was higher in the 0/100 (no dioxane) case (38%) than in the 99/1 (high dioxane) case (25%). However, gel electrophoresis indicated degradation of pDNA in the “no dioxane” mixture, whereas pDNA retained its structural integrity following SA1 in the high dioxane mixture. For the studies described below, a 70/30 dioxane/acetic acid (v/v) mixture, which gave FE values comparable to those in the “no dioxane” case but without significant pDNA degradation or loss of functionality (vida infra), was selected.

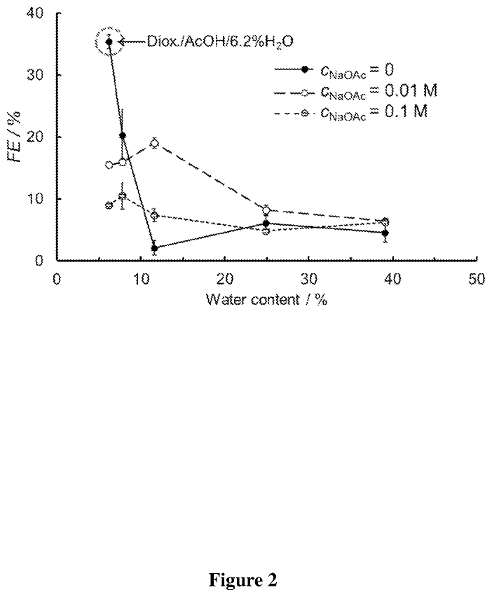

While holding the dioxane/acetic acid ratio constant at 70/30 (v/v), we next investigated the effects of water content and salt addition (sodium acetate) on FE (FIG. 2). In the absence of salt, we found that SA1 mixtures required a minimum water content of ˜6 wt %, below which pDNA was found to precipitate. As the water content was increased from 6.2 to 11.6 wt % t (solid black circle data points, C_{NaOAc}=0, 2), FE values dropped sharply (from 35% to less than 5%). To investigate the possibility that this drop in FE was associated with the formation of regular micelles with PCL cores and P2VP coronae above the critical water content (cwc) of the PCL blocks, we carried out a cwc determination of the copolymer/pDNA SA1 mixture solution using static light scattering (SLS); these results reveal the cwc to be ˜25 wt % well above the water contents associated with the sharp drop in FE (FIG. 2) such that PCL-b-P2VP micellization cannot explain the observation in this study.

When salt was added to the SA1 mixture, we found a general decrease in FE values, especially at low water contents. For the lowest water content (6.2 wt %), FE values were found to decrease sharply from 35% with no salt (solid black circle data points, C_{NaOAc}=0, FIG. 2), to 15% (c_{NaOAc}=0.01 M, open circle, long dash data points, FIG. 2) to 9% (c_{NaOAc}=0.1 M, open circle, short dash data points, FIG. 2). This effect can be attributed to the screening of electrostatic attractive interactions between negative phosphate groups on the pDNA and positive pyridinium groups on the P2VP blocks, which increases with the ionic strength of the mixture. Based on the maximum FE value shown in FIG. 2 (38%, open circle, long dash dashed circle), we chose the SA1 conditions of 70/30 dioxane/acetic acid (v/v), 6.2 wt % water, and no salt added for the remaining experiments described below.

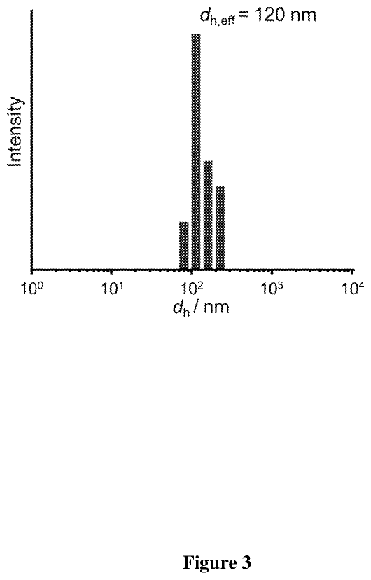

Characterization of PCL-pDNA Intermediate. Following the SA1 step, block ionomer micelles consisting of a PCL shell layer and a pDNA-P2VP polyplex core are designated PCL-pDNA. Before proceeding with the SA2 step, the size and pDNA integrity of the PCL-pDNA intermediate were characterized (FIG. 3). First, the effective hydrodynamic size distribution in the SA1 solvent mixture was determined by dynamic light scattering (DLS, FIG. 3). The resulting CONTIN size distribution reveals a single-particle population; the corresponding cumulant analysis gives a mean effective hydrodynamic diameter of d_{h,eff}=120±8 nm, which includes both the condensed pDNA-P2VP polyplex core and the expanded PCL shell (FIG. 3).

We next investigated the structural integrity of pDNA following the optimized SA1 process. Before structural analysis by gel electrophoresis, pDNA was released from the block ionomer micelles. To do this, functionalized pDNA was first isolated from the SA1 mixture by centrifugation then dispersed in chloroform, following the same procedure used for FE determination. Next, a deionized (DI) water layer was placed on top of the chloroform dispersion, followed by the addition of pyridine to the chloroform layer. The two-phase mixture was then stirred for 3 h, leading to deprotonation of P2VP pyridinium groups by the added pyridine; as a result, pDNA was released from the block ionomer micelles and extracted into the aqueous phase.

The pDNA that had been released from PCL-pDNA was run on the gel alongside an untreated pDNA standard and a control sample produced by stirring the two-phase mixture for only 30 min following pyridine addition. The presence of pDNA bands in the PCL-pDNA lane but not in the control sample lane suggests that 30 min stirring with pyridine was not sufficient to release pDNA from the block ionomer micelles although 3 h stirring was sufficient. The two main bands of the untreated pDNA standard, attributed to open circular (1) and supercoiled (2) forms, are also observed for pDNA released from PCL-pDNA. This indicates that the SA1 step did not degrade the functionalized pDNA. Accordingly, both the untreated pDNA standard lane and the PCL-pDNA lane showed bands associated with open circular (1) and supercoiled (2) forms, confirming that structural integrity of pDNA is retained during SAL.

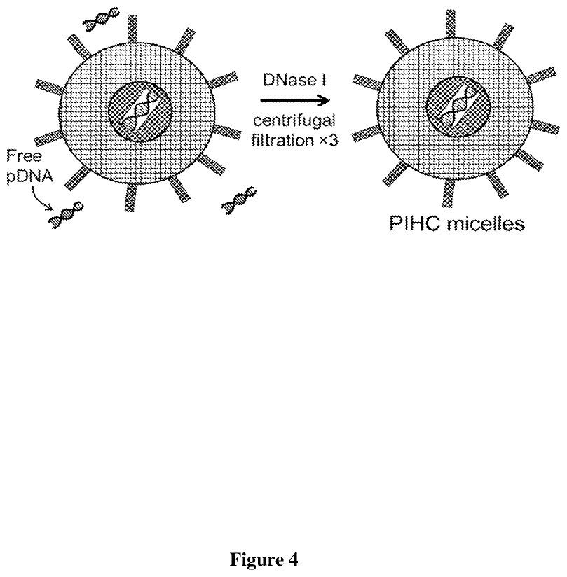

Formation of PIHC Micelles from the Self-Assembly of PCL-pDNA with PCL-b-PEG (SA2 Step). Having confirmed the incorporation and integrity of pDNA within the intermediate PCL-pDNA (FIG. 1A), we proceeded with the second self-assembly step. First, a dioxane solution containing PCL-b-PEG chains was added to the SA1 mixture. Then, water was added to above the critical water content using either the bulk or microfluidic method to initiate SA2 via co-condensation of PCL chains on PCL-pDNA and PCL-b-PEG components (FIG. 1B). The cwc of PCL-b-PEG alone (no PCL-pDNA) was determined to be 19.2±0.3 wt % at the solvent composition and copolymer concentration in the microfluidic channels, well below the water content (˜33 wt %) of PIHC micelle formation. This microprecipitation process formed the desired structures, with cores containing the pDNA polyplex surrounded by a protecting matrix of condensed hydrophobic PCL chains. The resulting dispersions were dialyzed against DI water to remove organic solvents. Finally, unencapsulated pDNA was removed from the sample by a process of DNase I digestion, followed by several cycles of centrifugal filtration and washing (FIG. 4). The final particles following removal of unencapsulated pDNA are designated PIHC micelles.

We have previously demonstrated significant effects of on-chip shear forces in two-phase microfluidic reactors such as the one applied here on the structure and function of polymer-based materials for nanomedicine materials.³⁷⁻⁴³ Therefore, we were interested in comparing PIHC micelle samples prepared by the bulk and microfluidic SA2 processes in terms of their encapsulation efficiencies (EE), size distributions, and morphologies.

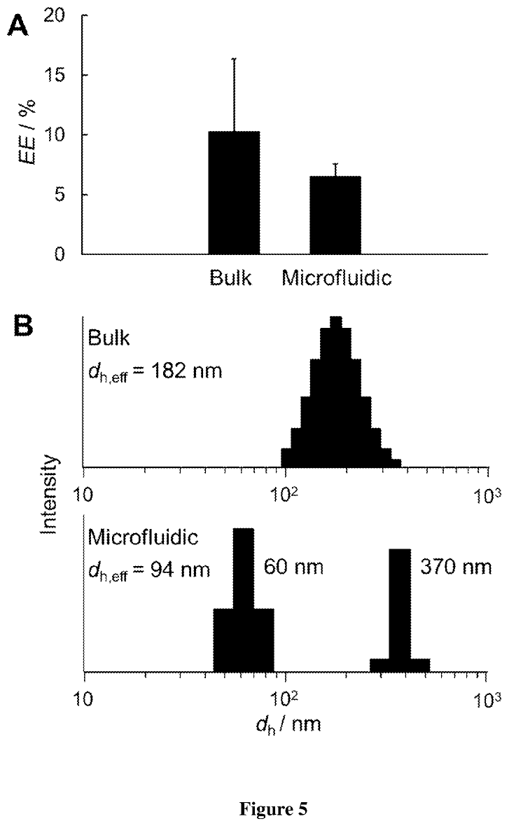

Encapsulation efficiencies (EE), determined from phosphate analysis of PIHC micelles, describe the percentage of total initial pDNA (45 μg) that was encapsulated in the hydrophobic cores following both SA1 and SA2 steps. The mean EE values shown for bulk (10.7%) and microfluidic (6.5%) methods in FIG. 5A were determined from triplicate preparations; despite the slightly higher mean EE value obtained using the bulk method, the variability of the method was significantly higher compared to the microfluidic approach. We attribute the greater reproducibility in EE values using the microfluidic method to improved control over mixing in microfluidic channels.⁴⁴ In our determination of EE, we make the assumption that all pDNA that was not encapsulated in hydrophobic cores was removed in the process of DNase I digestion, centrifugal filtration, and washing. To test this assumption, we carried out a phosphate assay of the negative control sample, which was prepared using the same steps, copolymers, and reagents as the PIHC micelle samples, except with the pDNA added after the SA2 step, such that the pDNA would remain outside the hydrophobic cores during DNase I digestion. No detectable phosphate was measured in the negative control, confirming that pDNA in both PIHC micelle samples was protected from DNase I digestion by its location within the hydrophobic cores of the micelles.

In 5B, CONTIN size distributions and mean effective hydrodynamic diameters, d_{h,eff}, from DLS data were compared for bulk and microfluidic PIHC micelle preparations. In both cases, measured d_{h,eff} values (d_{h,eff}=182±2 nm for bulk and d_{h,eff}=94±3 nm for microfluidic preparations) included the condensed PCL cores of PIHC micelles along with the solubilized PEG shells. Along with the larger mean size of thebulk sample, CONTIN analysis revealed that bulk PIHC micelle formation gave rise to a single broad size distribution (FIG. 5B, top), compared to microfluidic formation, which resulted in two separate populations of smaller (˜60 nm) and larger (˜370 nm) particles (5B, bottom). The two populations in the microfluidic CONTIN size distribution (5B, bottom) were similar in intensity; however, these distributions were weighted according to a scattered light intensity, which strongly emphasizes larger particles in the distribution, suggesting that the number of ˜60 nm particles strongly dominated over the number of ˜370 nm particles. In fact, when the microfluidic CONTIN result in 5B was converted to a number-weighted distribution, the larger particle population disappeared and only a single population of smaller particles was observed.

Transmission electron microscopy (TEM) images of the corresponding bulk and microfluidic samples supported the DLS analysis. Within the images, the uranyl acetate staining agent selectively bonded to the PEG coronae such that PCL cores appear bright. In the bulk sample, a broad distribution of irregularly shaped aggregates was observed. In contrast, the microfluidic sample was comprised mainly of low-polydispersity spherical PIHC micelles with ˜30 nm cores, along with a small number of large irregular aggregates. The smaller size of spherical PCL cores determined by TEM (˜30 nm) compared to hydrodynamic diameters determined by DLS (˜60 nm) is consistent with a hydrated PEG layer of ˜15 nm surrounding PIHC micelles; this is about half the contour length of 5 k PEG chains (31.8 nm), suggesting that coronal chains are ˜50% extended.

Without being bound to a particular hypothesis, the contrasting size distributions and morphologies of the bulk and microfluidic PIHC micelle populations may be explained by considering different SA2 self-assembly environments in the two cases. In the bulk SA2 case, water was added dropwise to the copolymer/pDNA mixture, with slow diffusional mixing leading to chemical heterogeneity throughout the sample as self-assembly occurs; this forms a broad distribution of aggregates that become kinetically trapped by slow chain dynamics as the water content increases. In contrast, the fast mixing⁴⁴ between water and the copolymer/pDNA stream in the microfluidic mixing channel results in a homogenous chemical environment during the SA2 process, leading to a uniform population of spherical PIHC micelles. The subsequent exposure to high shear “hot spots”⁴⁵ in the processing channel gives rise to an interplay of shear-induced coalescence and shear-induced breakup,⁴⁶ which could explain the presence of some larger nonspherical aggregates within the microfluidic sample. Another possibility is that the minor population of larger aggregates forms from excess individual PCL-b-PEG chains that do not coassemble with PCL-pDNA in the SA2 process (FIG. 1B). Based on the predominance of small, low-polydispersity spheres, which are favorable for cell uptake⁴³ and targeting,¹² in addition to the greater reproducibility of pDNA encapsulation, we selected the microfluidic preparation method for further analysis, including in vitro cell experiments.

We can estimate the pDNA occupancy of the micelles in the microfluidic PIHC micelle sample by making a few simplifying assumptions. First, based on the dominant spherical micelle population in TEM images, we assume that all of the PCL from both copolymer components assembles into 30 nm spherical cores; we calculate the core volume (V=4/3πr³) to be V_{core}=1.4×10⁻¹⁷ mL. Then, using a density value of 1.14 g/mL for PCL cores,⁴⁷ we calculate the mass of the cores to m core=1.6×10⁻¹⁷ g. We next assume that no copolymer is lost in the self-assembly, dialysis, and centrifugal filtration steps; this assumption, together with the total PCL mass added in both SA1 (0.100 mg) and SA2 (0.015 mg) steps (m_{PCL}=0.115×10⁻³ g), allows us to estimate the number of spherical micelles in the sample: N_{mic}=m_{PCL}/m_{core}=7×10¹². Next, based on the initial mass of pDNA (45 μg) and the mean EE value for the microfluidic sample (6.5%), we determine that the total pDNA mass in the PIHC micelles to be m_{pDNA}=2.92×10⁻⁶ g. Then, using this value and the molecular weight of pUC18 (M_{pDNA}=1.75×10⁶ g/mol), we estimate the number of encapsulated plasmids in the sample using

$${N}_{pDNA} = \frac{{m}_{pDNA}}{{M}_{pDNA}} \times {N}_{A} , where {N}_{A} = 6.02 \times {10}^{23} .$$

This calculation gives N_{pDNA}=1×10¹², which, together with N_{mic}=7×10¹², yields a pDNA occupancy of 0.14, or about one plasmid per seven micelles. This value should only be taken as a rough estimate; the pDNA occupancy may be much higher, considering possible polymer losses and/or partitioning of single copolymer chains into larger nonspherical aggregates. As described below, laser scanning confocal fluorescence microscopy (LSCFM) imaging of PIHC micelles with two different dye labels on pDNA and PCL components confirmed that not all micelles contain pDNA but that all pDNA is associated with micelles.

To confirm the structural integrity of pDNA following SA2 self-assembly, we released encapsulated pDNA in a two-step process. First, acetonitrile was added to the microfluidic dispersion, leading to PCL dissolution and concomitant PIHC micelle breakup. Next, the constituent block ionomer polyplexes were recovered by centrifugation, dispersed in chloroform, then disassembled by addition of pyridine and extraction of free pDNA into an aqueous layer. The resulting pDNA was run on a gel with the untreated pDNA standard and a 1 kb DNA ladder. Similar to pDNA release following the SA1 step, we found that the two main bands of the untreated pDNA standard, attributed to open circular (1) and supercoiled (2) forms, are present following SA2 self-assembly. This confirms that neither SA1 nor SA2 steps resulted in degradation of encapsulated pDNA.

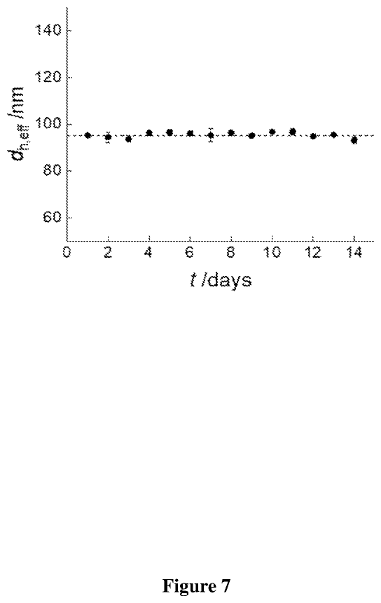

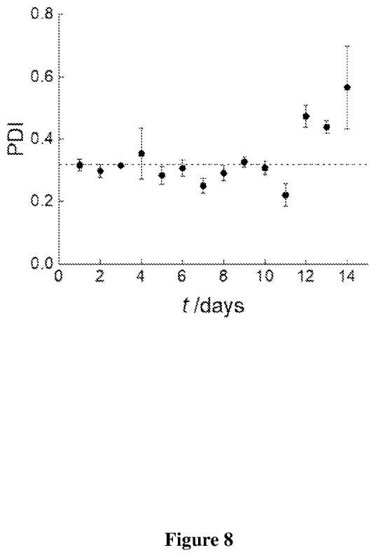

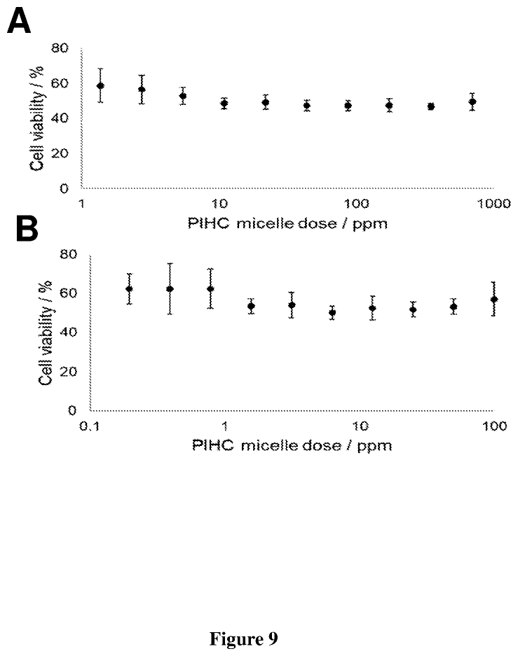

The surface charge and long-term stability of a microfluidic PIHC micelle sample were also investigated. Measured ζ potential for the microfluidic dispersion was close to zero (ζ=−2±1 mV), consistent with neutral PEG coronal chains and encapsulation of charged pDNA within the PCL cores. The colloidal stability of PIHC micelles was determined by monitoring d_{h,eff} values of the microfluidic dispersion over 2 weeks; in between daily DLS measurements, the sample was stored in a sealed vial in the dark at 4° C. Compared to the initial (t=1 day) measurement (d_{h,eff}=95.2±0.6 nm, polydispersity index (PDI)=0.32±0.02), the microfluidic sample showed no significant variation in size over 2 weeks and no significant increase in PDI until 12 days (FIGS. 7 and 8), indicating good long-term colloidal stability. This suggests that the PEG coronal layer provides colloidal stabilization of PIHC micelles despite its neutral charge, presumably via repulsive steric interactions between micelles. The reported critical micelle concentration for a PCL-b-PEG copolymer with identical block lengths has been reported in the literature to be 67 mg/L,⁴⁸ further suggesting good thermodynamic stability for PIHC micelles.

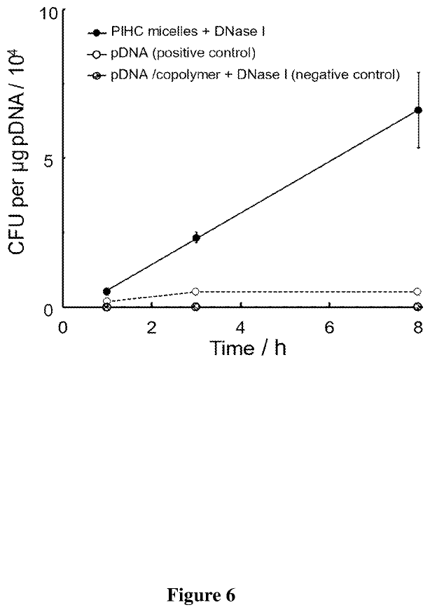

Bacterial Cell Transformation. To investigate the functionality and timed release of pDNA from PIHC micelles, we carried out transformation experiments on competent Escherichia coli K12 cells. FIG. 6 shows transformation efficiencies as functions of incubation time for the microfluidic PIHC micelle sample along with the negative and positive controls. The negative control consisted of nanoparticles produced in the same manner as PIHC micelle formation except with pDNA added after SA2 assembly, such that pDNA was not encapsulated during DNase I exposure. The positive control consisted of free pDNA that was exposed to the same solvents, dialysis, and centrifugal filtration as encapsulated pDNA except without the addition of copolymers and without DNase I exposure.

As shown in FIG. 6, no transformants were observed for the negative control at any incubation time up to 8 h (open circle datapoints, bottom). This indicates that DNase I exposure precluded subsequent transformation when pDNA was not encapsulated within the hydrophobic cores of the accompanying copolymer nanoparticles; we attribute this result to complete degradation of unencapsulated pDNA by DNase I, as supported by phosphate assay results for the negative control. On the other hand, significant bacterial transformation was found for the positive control (open circle data points, with dashed linear trendline, middle), which increased between 1 and 3 h of incubation but then leveled off with no further increase measured after 8 h of incubation. This result confirms the functionality of free pDNA without DNAase I exposure; it also indicates some time dependence of bacterial transformation by free pDNA in the first 3 h of incubation but negligible time dependence in the subsequent 5 h of incubation. Compared to the positive control, the PIHC micelle sample (closed black circle data points, top) showed dramatically higher transformation efficiencies at all time points (up to 12× higher after 8 h of incubation). It also showed a much stronger positive time dependence over the entire 8 h incubation period; the three time points fit well to a linear trendline.

Without being bound to a particular hypothesis, the higher transformation efficiencies of PIHC micelles compared to the free plasmid may be related to a lowering of energy barriers for uptake by competent cells, due to a combination of lower charge and smaller hydrodynamic diameter (˜60 nm) of the spherical micelles compared to uncondensed pUC18 (140-160 nm).⁴⁹ A similar ˜10-fold increase in transformation efficiencies of competent E. coli was previously found for pDNA complexed to Fe₃O₄ nanoparticles, although the authors did not propose a mechanism in that study.⁵⁰ Additionally, and without being bound to a particular hypothesis, the observed increase in transformation over 8 h, in contrast to free pDNA, which showed no increase in activity after 3 h, may be attributed to the location of the pDNA-P2VP polyplex within the micellar PCL cores, such that pDNA release within the cell requires hydrolytic degradation of the surrounding matrix of PCL chains.^{37,51}

Uptake of PIHC Micelles into MDA-MB-231 Cells

Finally, we investigated the uptake of PIHC micelles by MDA-MB-231 (human breast cancer) cells using fluorescence microscopy. The PIHC micelles used for these experiments were labeled with two different fluorescent dyes during synthesis: (1) 4′,6-diamidino-2-phenylindole (DAPI) is a DNA-intercalating dye that was used to selectively label pDNA and (2) 1′-dioctadecyl-3,3,3′,3′-tetramethylindocarbo-cyanine perchlorate (DiI) is a hydrophobic dye that was used to label PCL cores. The cytosolic regions of MDA-MB-231 cells were also labeled with fluorescein diacetate. A comparison of the blue fluorescence from DAPI-labeled pDNA and the red fluorescence from DiI-labeled micelles confirmed that plasmid DNA was successfully delivered to many of the target cells. DNA delivery appears to be stochastic, with some cells receiving considerably greater amounts of DNA than others, and some cells containing DiI-labeled PCL without significant quantities of DAPI-labeled pDNA. These findings are consistent with the DNA occupancy calculation above, which estimated a loading of approximately one plasmid per seven micelles. They are also consistent with LSCFM imaging of the same DAPI/DiI-labeled PIHC micelles deposited on a glass substrate without cells, which confirms that while many micelles do not contain pDNA, all pDNA is originally colocalized with micelle cores. We also note that all cells that were positive for pDNA (blue fluorescence) also showed the presence of lipophilic polymer (red fluorescence) consistent with the proposed PIHC-based delivery mechanism.

An overlay of blue fluorescence from DAPI-labeled pDNA with red fluorescence from DiI-labeled micelles reveals a lack of colocalization between the two species, which suggests that at least some of the pDNA has escaped the delivery vehicle during the 4 h incubation period. Separate cytotoxicity experiments also revealed that unlabeled PIHC micelles (i.e., without fluorescent dyes) showed no significant toxicity toward MDA-MB-231 cells over 4 orders of magnitude of dosing levels (FIGS. 8A-B).

### REFERENCES

(1) Dunbar, C. E.; High, K. A.; Joung, J. K.; Kohn, D. B.; Ozawa, K.; Sadelain, M. Gene Therapy Comes of Age. Science 2018, 359, No. eaan4672.
(2) Hardee, C. L.; Arevalo-Soliz, L. M.; Hornstein, B. D.; Zechiedrich, L. Advances in Non-Viral DNA Vectors for Gene Therapy. Genes 2017, 8, No. 65.
(3) Xiang, Y. G.; Oo, N. N. L.; Lee, J. P.; Li, Z. B.; Loh, X. J. Recent Development of Synthetic Nonviral Systems for Sustained Gene Delivery. Drug Discovery Today 2017, 22, 1318-1335.
(4) Pereira-Silva, M.; Jarak, I.; Alvarez-Lorenzo, C.; Concheiro, A.; Santos, A. C.; Veiga, F.; Figueiras, A. Micelleplexes as Nucleic Acid Delivery Systems for Cancer-Targeted Therapies. J. Controlled Release 2020, 323, 442-462.
(5) Li, S.; Huang, L. Nonviral Gene Therapy: Promises and Challenges. Gene Ther. 2000, 7, 31-34.
(6) Li, L.; He, Z.-Y.; Wei, X.-W.; Gao, G.-P.; Wei, Y.-Q. Challenges in CRISPR/CAS9 Delivery: Potential Roles of Nonviral Vectors. Hum. Gene Ther. 2015, 26, 452-462.
(7) Bono, N.; Ponti, F.; Mantovani, D.; Candiani, G. Non-Viral in Vitro Gene Delivery: It is Now Time to Set the Bar! Pharmaceutics 2020, 12, 183-205.
(8) Li, J.; Kataoka, K. Chemo-physical Strategies to Advance the in Vivo Functionality of Targeted Nanomedicine: The Next Generation. J. Am. Chem. Soc. 2021, 143, 538-559.
(9) Santana-Armas, M. L.; Tros de Ilarduya, C. Strategies for Cancer Gene-Delivery Improvement by Non-Viral Vectors. Int. J. Pharm. 2021, 596, No. 120291.
(10) Song, H.; Hart, S. L.; Du, Z. Assembly Strategy of Liposome and Polymer Systems for siRNA Delivery. Int. J. Pharm. 2021, 592, No. 120033.
(11) Zu, H.; Gao, D. Non-viral Vectors in Gene Therapy: Recent Development, Challenges, and Prospects. AAPS J. 2021, 23, No. 78.
(12) Elsabahy, M.; Wooley, K. L. Design of Polymeric Nanoparticles for Biomedical Delivery Applications. Chem. Soc. Rev. 2012, 41, 2545-2561.
(13) Fliervoet, L. A. L.; Engbersen, J. F. J.; Schiffelers, R. M.; Hennink, W. E.; Vermonden, T. Polymers and Hydrogels for Local Nucleic Acid Delivery. J. Mater. Chem. B 2018, 6, 5651-5670.
(14) Cabral, H.; Miyata, K.; Osada, K.; Kataoka, K. Block Copolymer Micelles in Nanomedicine Applications. Chem. Rev. 2018, 118, 6844-6892.
(15) Chen, C. K.; Huang, P. K.; Law, W. C.; Chu, C. H.; Chen, N. T.; Lo, L. W. Biodegradable Polymers for Gene-Delivery Applications. Int. J. Nanomed. 2020, 15, 2131-2150.
(16) Salameh, J. W.; Zhou, L.; Ward, S. M.; Santa Chalarca, C. F.; Emrick, T.; Figueiredo, M. L. Polymer-Mediated Gene Therapy: Recent Advances and Merging of Delivery Techniques. Wiley Interdiscip. Rev.: Nanomed. Nanobiotechnol. 2020, 12, No. e1598.
(17) Uchida, S.; Kataoka, K. Design Concepts of Polyplex Micelles for In Vivo Therapeutic Delivery of Plasmid DNA and Messenger RNA. J. Biomed. Mater. Res., Part A 2019, 107, 978-990.
(18) Kargaard, A.; Sluijter, J. P. G.; Klumperman, B. Polymeric siRNA Gene Delivery: Transfection Efficiency Versus Cytotoxicity. J. Controlled Release 2019, 316, 263-291.
(19) Kim, H. J.; Kim, A.; Miyata, K.; Kataoka, K. Recent Progress in Development of siRNA Delivery Vehicles for Cancer Therapy. Adv. Drug Delivery Rev. 2016, 104, 61-77.
(20) Kim, H. J.; Ogura, S.; Otabe, T.; Kamegawa, R.; Sato, M.; Kataoka, K.; Miyata, K. Fine-Tuning of Hydrophobicity in Amphiphilic Polyaspartamide Derivatives for Rapid and Transient Expression of Messenger RNA Directed Toward Genome Engineering in Brain. ACS Cent. Sci. 2019, 5, 1866-1875.
(21) Endres, T. K.; Beck-Broichsitter, M.; Samsonova, O.; Renette, T.; Kissel, T. H. Self-Assembled Biodegradable Amphiphilic PEGPCL-IPEI Triblock Copolymers at the Borderline between Micelles and Nanoparticles Designed for Drug and Gene Delivery. Biomaterials 2011, 32, 7721-7731.
(22) Cao, N.; Cheng, D.; Zou, S. Y.; Ai, H.; Gao, J. M.; Shuai, X. T. The Synergistic Effect of Hierarchical Assemblies of siRNA and Chemotherapeutic Drugs Co-delivered into Hepatic Cancer Cells. Biomaterials 2011, 32, 2222-2232.
(23) Cheng, D.; Cao, N.; Chen, J. F.; Yu, X. S.; Shuai, X. T. Multifunctional Nanocarrier Mediated Co-delivery of Doxorubicin and siRNA for Synergistic Enhancement of Glioma Apoptosis in Rat. Biomaterials 2012, 33, 1170-1179.
(24) Endres, T.; Zheng, M. Y.; Kilic, A.; Turowska, A.; Beck-Broichsitter, M.; Renz, H.; Merkel, O. M.; Kissel, T. Amphiphilic Biodegradable PEG-PCL-PEI Triblock Copolymers for FRET Capable in Vitro and in Vivo Delivery of siRNA and Quantum Dots. Mol. Pharmaceutics 2014, 11, 1273-1281.
(25) Wu, Y.; Zhang, Y.; Zhang, W.; Sun, C. L.; Wu, J. Z.; Tang, J. H. Reversing of Multidrug Resistance Breast Cancer by Co-delivery of Pgp siRNA and Doxorubicin via Folic Acid-Modified Core-Shell Nanomicelles. Colloid Surf., B 2016, 138, 60-69.
(26) Lin, D. S.; Jiang, Q.; Cheng, Q.; Huang, Y. Y.; Huang, P. S.; Han, S. C.; Guo, S. T.; Liang, Z. C.; Dong, A. J. Polycation-Detachable Nanoparticles Self-Assembled from mPEG-PCL-g-SSPDMAEMA for In Vitro and In Vivo siRNA Delivery. Acta Biomater. 2013, 9, 7746-7757.
(27) Lee, S. Y.; Yang, C. Y.; Peng, C. L.; Wei, M. F.; Chen, K. C.; Yao, C. J.; Shieh, M. J. A Theranostic Micelleplex Co-delivering SN-38 and VEGF siRNA for Colorectal Cancer Therapy. Biomaterials 2016, 86, 92-105.
(28) Osawa, S.; Osada, K.; Hiki, S.; Dirisala, A.; Ishii, T.; Kataoka, K. Polyplex Micelles with Double-Protective Compartments of Hydrophilic Shell and Thermoswitchable Palisade of Poly(oxazoline)-Based Block Copolymers for Promoted Gene Transfection. Biomacromolecules 2016, 17, 354-361.
(29) Chen, M. H.; Zhang, Y.; Chen, Z. J.; Xie, S. Z.; Luo, X. M.; Li, X. H. Synergistic Antitumor Efficacy of Redox and pH Dually Responsive Micelleplexes for Co-delivery of Camptothecin and Genes. Acta Biomater. 2017, 49, 444-455.
(30) Grossen, P.; Witzigmann, D.; Sieber, S.; Huwyler, J. PEG-PCL based Nanomedicines: A Biodegradable Drug Delivery System and its Application. J. Controlled Release 2017, 260, 46-60.
(31) Huff, J.; Grant, B.; Penning, C.; Sullivan, K. Optimization of Routine Transformation of Escherichia coli with Plasmid DNA. BioTechniques 1990, 9, 570-572 574, 576.
(32) West, S.; Schweizer, H.; Dall, C.; Sample, A.; Runyen-Janecky, L. Construction of Improved Escherichia-Pseudomonas Shuttle Vectors Derived from pUC18/19 and Sequence of the Region Required for their Replication in Pseudomonas Aeruginosa. Gene 1994, 148, 81-86.
(33) Yokoya, A.; Shikazono, N.; Fujii, K.; Urushibara, A.; Akamatsu, K.; Watanabe, R. DNA Damage Induced by the Direct Effect of Radiation. Radiat. Phys. Chem. 2008, 77, 1280-1285.
(34) Yoshida, N.; Sato, M. Plasmid Uptake by Bacteria: a Comparison of Methods and Efficiencies. Appl. Microbiol. Biotechnol. 2009, 83, 791-798.
(35) Nazemi, A.; Boott, C. E.; Lunn, D. J.; Gwyther, J.; Hayward, D. W.; Richardson, R. M.; Winnik, M. A.; Manners, I. Monodisperse Cylindrical Micelles and Block Comicelles of Controlled Length in Aqueous Media. J. Am. Chem. Soc. 2016, 138, 4484-4493.
(36) Danielsen, S.; Maurstad, G.; Stokke, B. T. DNA-Polycation Complexation and Polyplex Stability in the Presence of Competing Polyanions. Biopolymers 2005, 77, 86-97.
(37) Bains, A.; Wulff, J. E.; Moffitt, M. G. Microfluidic Synthesis of Dye-Loaded Polycaprolactone-block-poly (ethylene oxide) Nano-particles: Insights into Flow-Directed Loading and In Vitro Release for Drug Delivery. J. Colloid Interface Sci. 2016, 475, 136-148.
(38) Bains, A.; Cao, Y. M.; Kly, S.; Wulff, J. E.; Moffitt, M. G. Controlling Structure and Function of Polymeric Drug Delivery Nanoparticles using Microfluidics. Mol. Pharmaceutics 2017, 14, 2595-2606.
(39) Cao, Y.; Silverman, L.; Lu, C.; Hof, R.; Wulff, J. E.; Moffitt, M. G. Microfluidic Manufacturing of SN-38-Loaded Polymer Nanoparticles with Shear Processing Control of Drug Delivery Properties. Mol. Pharmaceutics 2019, 16, 96-107.
(40) Chen, R.; Wulff, J. E.; Moffitt, M. G. Microfluidic Processing Approach to Controlling Drug Delivery Properties of Curcumin-Loaded Block Copolymer Nanoparticles. Mol. Pharmaceutics 2018, 15, 4517-4528.
(41) Jensen, D.; Cao, Y.; Lu, C.; Wulff, J. E.; Moffitt, M. G. Microfluidic Encapsulation of SN-38 in Block Copolymer Nanoparticles: Effect of Hydrophobic Block Composition on Loading and Release Properties. Can. J. Chem. 2019, 97, 337-343.
(42) Huang, Y.; Moini Jazani, A.; Howell, E. P.; Oh, J. K.; Moffitt, M. G. Controlled Microfluidic Synthesis of Biological Stimuli-Responsive Polymer Nanoparticles. ACS Appl. Mater. Interfaces 2020, 12, 177-190.
(43) Huang, Y.; Jazani, A. M.; Howell, E. P.; Reynolds, L. A.; Oh, J. K.; Moffitt, M. G. Microfluidic Shear Processing Control of Biological Reduction Stimuli-Responsive Polymer Nanoparticles for Drug Delivery. ACS Biomater. Sci. Eng. 2020, 6, 5069-5083.
(44) Schabas, G.; Wang, C. W.; Oskooei, A.; Yusuf, H.; Moffitt, M. G.; Sinton, D. Formation and Shear-Induced Processing of Quantum Dot Colloidal Assemblies in a Multiphase Microfluidic Chip. Langmuir 2008, 24, 10596-10603.
(45) Wang, C. W.; Oskooei, A.; Sinton, D.; Moffitt, M. G. Controlled Self-Assembly of Quantum Dot-Block Copolymer Colloids in Multiphase Microfluidic Reactors. Langmuir 2010, 26, 716-723.
(46) Wang, C. W.; Sinton, D.; Moffitt, M. G. Morphological Control via Chemical and Shear Forces in Block Copolymer Self-Assembly in the Lab-on-Chip. ACS Nano 2013, 7, 1424-1436.
(47) Rosa, D.; Neto, I. C.; Calil, M.; Pedroso, A.; Fonseca, C.; Neves, S. Evaluation of the Thermal and Mechanical Properties of Poly(ε-caprolactone), Low-Density Polyethylene, and their Blends. J. Appl. Polym. Sci. 2004, 91, 3909-3914.
(48) Theerasilp, M.; Nasongkla, N. Comparative Studies of Poly(ε-caprolactone) and Poly(D,L-lactide) as Core Materials of Polymeric Micelles. J. Microencapsulation 2013, 30, 390-397.
(49) Tsoi, M.; Do, T. T.; Tang, V.; Aguilera, J. A.; Perry, C. C.; Milligan, J. R. Characterization of Condensed Plasmid DNA Models for Studying the Direct Effect of Ionizing Radiation. Biophys. Chem. 2010, 147, 104-110.
(50) Saei, A. A.; Barzegari, A.; Majd, M. H.; Asgari, D.; Omidi, Y. Fe3O4 Nanoparticles Engineered for Plasmid DNA Delivery to Escherichia coli. J. Nanopart. Res. 2014, 16, No. 2521.
(51) Geng, Y.; Discher, D. E. Hydrolytic Degradation of Poly-(ethylene oxide)-block polycaprolactone Worm Micelles. J. Am. Chem. Soc. 2005, 127, 12780-12781.
(52) Bartlett, G. R. Phosphorus Assay in Column Chromatography. J. Biol. Chem. 1959, 234, 466-468.
(53) Maples, R. E. Petroleum Refinery Process Economics; Pennwell Books, 2000.
(54) Reis, J. C. R.; Lampreia, I. M.; Santos, A. F.; Moita, M. L. C.; Douhret, G. Refractive Index of Liquid Mixtures: Theory and Experiment. ChemPhysChem 2010, 11, 3722-3733.

All citations are hereby incorporated by reference.

The present invention has been described with regard to one or more embodiments. However, it will be apparent to persons skilled in the art that a number of variations and modifications can be made without departing from the scope of the invention as defined in the claims.

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. “About”, as used herein, means a deviation of +/−10%. In the specification, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

What is claimed is:

1. A micelle comprising:
i) a first block copolymer comprising a polycationic segment and a first hydrophobic segment; and
a nucleic acid molecule reversibly bound to the polycationic segment of the block copolymer; and
ii) a second block copolymer comprising a second hydrophobic segment and a water-soluble segment,
wherein the first and second hydrophobic segments form a matrix of condensed hydrophobic chains and form a core together with the nucleic acid molecule; and
wherein the water-soluble segment forms a hydrophilic coronal layer that contains the core.

2. The micelle of claim 1 wherein the first block copolymer is poly(ε-caprolactone)-block-poly(2-vinyl pyridine).

3. The micelle of claim 1 wherein the second block copolymer is poly(ε-caprolactone)-block-poly(ethylene glycol).

4. The micelle of claim 1 wherein the micelle comprises one or more cores.

5. The micelle of claim 1 wherein the nucleic acid molecule is DNA or RNA.

6. A method for preparing a micelle comprising:
contacting a nucleic acid molecule with a first block copolymer comprising a polycationic segment and a first hydrophobic segment under conditions suitable for reversible binding to form a polyplex; and
contacting the polyplex with a block copolymer comprising the hydrophobic segment and a water-soluble segment under conditions suitable for condensation of the first and second hydrophobic segments,
wherein the condensation results in the formation of the micelle.

7. The method of claim 6 wherein the first block copolymer is poly(ε-caprolactone)-block-poly(2-vinyl pyridine).

8. The method of claim 6 wherein the second block copolymer is poly(ε-caprolactone)-block-poly(ethylene glycol).

9. The method of claim 6 wherein the micelle comprises one or more cores.

10. The method of claim 6 wherein the nucleic acid molecule is DNA or RNA.

11. A method of transforming or transfecting a cell, or of delivering a nucleic acid molecule to a cell, comprising contacting the cell with the micelle of claim 1.

12. The method of claim 11 wherein the first block copolymer is poly(ε-caprolactone)-block-poly(2-vinyl pyridine).

13. The method of claim 11 wherein the second block copolymer is poly(ε-caprolactone)-block-poly(ethylene glycol).

14. The method of claim 11 wherein the micelle comprises one or more cores.

15. The method of claim 11 wherein the nucleic acid molecule is DNA or RNA.

16. The method of claim 11 wherein the cell is a prokaryotic cell or a eukaryotic cell.

17. The method of claim 16 wherein the eukaryotic cell is in a tissue or organ.
